(19) United States
(12) Patent Application Publication
(10) Pub. No.: US 2013/0202766 A1
(43) Pub. Date: Aug. 8, 2013

(54) Composition, Methods and Devices for Reduction of Cells in a Volume of Matter Using Low Voltage High Electric Field (LVHEF) Electrical Energy

(71) Applicant: Elmedtech, LLC, San Francisco, CA (US)

(72) Inventors: Boris Rubinsky, El Cerrito, CA (US); Liel Rubinsky, El Cerrito, CA (US)

(73) Assignee: Elmedtech, LLC, San Francisco, CA (US)

(21) Appl. No.: 13/758,834

(22) Filed: Feb. 4, 2013

Related U.S. Application Data
(60) Provisional application No. 61/595,547, filed on Feb. 6, 2012.
(60) Provisional application No. 61/671,520, filed on Jul. 13, 2012.
(60) Provisional application No. 61/730,750, filed on Nov. 28, 2012.

(51) Int. Cl. A61L 2/03; A23L 3/32

(52) U.S. Cl. CPC A61L 2/03; A23L 3/32
USPC 426/590; 422/22; 422/186.04; 426/531; 99/485

(57) ABSTRACT

The present disclosure provides devices, systems, and methods for affecting cells using low voltage high electric fields (LVHEF). In one embodiment, the present disclosure provides for reduction of microbial contamination using low voltage high electric fields. The devices of the disclosure are generally capable of affecting cells in a portion of a volume of matter of interest using one or more arrangements of electrodes configured to generate high electric fields powered by low voltages (LVHEF). In one embodiment, the present disclosure provides for exposure of cells to a low voltage high electric field such that at least a portion of the cells in a portion of the volume of interest are killed. While only a portion of the matter is treated at a single time, the treatments are repeated. Over time, the portion of matter in the treated volume is mixed with untreated matter and re-treated with LVHEF until the entire volume of matter of interest is treated to the desired level. The voltage required to treat the volume of matter of interest with LVHEF is substantially lower than the voltage required for treating the volume of matter of interest through a single application of the high electric fields without mixing. In one aspect, electrodes are arranged in a co-planar configuration. The disclosure provides for a variety of applications and products, including consumer goods and pharmaceuticals.

This application claims priority under 35 USC §119(e) to U.S. Provisional Application 61/595,547, filed Feb. 6, 2012, U.S. Provisional Application 61/671,520, filed Jul. 13, 2012, and U.S. Provisional Application 61/730,750, filed Nov. 28, 2012, each of which are incorporated by reference in their entirety.

### BACKGROUND

Contamination by microorganisms is a recognized problem in numerous industries, ranging from food and beverage processing, drinking water decontamination, pharmaceutical and drug packaging to medical device sterilization and the like. Processes for control and reduction of microorganisms, while divergent in application, are commonly concerned with preventing the contamination of various mediums with microorganisms that may affect the quality of products and/or human health. Many types of microorganisms, ranging from bacteria to fungi, may cause adverse effects in different application settings. In the field, there have been various strategies, devices and products to control contamination ranging from physical means via filtration or heat, to chemical means via preservatives and other chemical additives.

While chemical additives such as preservatives have been effective in reducing microorganism contamination in a wide variety of media, from beverages to drugs, their use is not always optimal. Aside from added cost, preservatives can sometimes alter the taste of certain foods and beverages or cause allergic or toxic reactions in sensitive individuals, especially when added to drugs or medical products. Additionally, some microorganisms, particularly some bacteria and fungi and parasites, can live in media even in the presence of some chemical preservatives.

Another approach that has been studied is the reduction of microbial contaminants in a medium using electricity. Efforts have been focused on the reduction of microorganisms in a medium by exposing these contaminants to high voltage high strength electric fields generated uniformly throughout the entirety of the medium, exposing the entirety of the medium at the same time. Devices in the field generate high strength electric fields using high voltages to ensure killing of the microorganisms.

Some have attempted the reduction of microorganisms by non-thermal methods, whereby high voltage, high strength electric fields are applied to a medium in short pulses. These electric fields allow primarily for the irreversible electroporation of microorganisms, a process by which pores open in the membranes of cells, releasing cellular components and thus killing the cell. These high strength electric fields delivered in other forms, known as nanosecond pulses, can also produce damage to the intracellular components of a cell. The shortness of the pulse is thought to prevent the build-up of excessive heat in the medium. This use of high strength electric fields has been performed on devices that employ various types of opposed electrodes that generate a substantially homogeneous electric field throughout the entirety of the medium at one time. These devices have been configured to treat a range of media, from food and beverages such as milk and water to bodily fluids such as blood.

Despite these efforts, current devices have deficiencies in a variety of applications. Current devices requiring high strength electric fields for the production of irreversible electroporation in cells employ large voltages which typically require large or inconvenient power sources. In some instances, devices employing large voltages may limit applications of the device, both in effectiveness of treatment as well as the types of media to which it may be applied.

Thus, there remains a need for processes and devices for control and reduction of microorganisms with irreversible electroporation type electric fields that overcome one or more of these deficiencies. This invention addresses this need, and provides additional advantages as described below.

### SUMMARY OF THE INVENTION

The disclosure provides for methods for affecting cells in a volume of matter, where the method includes i) exposing a portion of the volume to one or more electric fields that are sufficient to kill at least a portion of exposed cells, ii) mixing the exposed portion of the volume with an unexposed portion of the volume to form a mixed volume, and iii) exposing a portion of the mixed volume to one or more electric fields that are sufficient to kill at least a portion of exposed cells, where the volume of matter is bounded by a surface having one or more electrodes.

The disclosure also provides for methods for reducing contamination by microorganisms in a volume of matter, the method comprising: i) exposing a portion of the volume to one or more electric fields that are sufficient to kill at least a portion of exposed microorganisms, ii) mixing the exposed portion of the volume with an unexposed portion of the volume to form a mixed volume, and iii) exposing a portion of the mixed volume to one or more electric fields that are sufficient to kill at least a portion of exposed microorganisms; wherein the volume of matter is bounded by a surface having one or more electrodes.

The disclosure further provides for devices, apparatuses, and systems for performing the methods as described herein. For example, in various aspects, the disclosure provides for a container, apparatus, or device configured to perform the methods described herein. In various embodiments, the container is further configured to enclose a food or beverage. In various embodiments, the container is further configured to enclose a solution or object selected from the group consisting of a pharmaceutical agent, a medical agent, and a medical device. In various embodiments, the device is configured as an insert in a container of food or beverage or drugs. In various embodiments, the methods, devices, and containers disclosed herein are used for cleaning contact lenses during temporary storage, such as for a few hours or overnight.

The disclosure also provides for compositions of matter treated with such methods, devices, apparatuses, and systems. In various embodiments, the matter to be treated is selected from a liquid, gas, heterogenous composition such as a solid or glass in a liquid, fluid, colloid, gel, aerosol, foam, emulsion, suspension, solution, and mixtures thereof. In various embodiments, the composition is selected from the group consisting of food, beverage, cosmetics, and pharmaceutical. In various embodiments, the matter is aqueous. In various embodiments, the composition of matter treated according to the disclosure has a taste, which is not detectably altered by the method when subjected to a human taste test. In various embodiments the matter has a medical function, such as a drug, and the treatment does not affect the medical function of the matter. In various embodiments, the composition of matter treated with methods as disclosed herein includes contact lens solution. In various embodiments, the composition of matter treated with methods as disclosed herein comprises a contact lens. In various embodiments, the composition of matter includes eye drops. In various embodiments, the composition is a composition for injection. In various embodiments, the composition is a macromolecule or protein.

With regard to the methods disclosed herein, the sequence of steps may occur continuously, sequentially, or sequentially-in-part and continuously-in-part. With regard to the mixing step, such mixing may occur through fluid movement. In various embodiments, the fluid movement is selected from the group consisting of: convection, forced convection, natural convection, mechanical agitation, vibration, stirring, electrical field driven flows, electrophoresis, dielectrophoresis, osmotic flow, electro-osmosis, turbulent flow, laminar flow, diffusion, and combinations thereof.

Any cells or microorganisms may be subjected to the methods of the disclosure. In various embodiments, cells are selected from microorganisms, unicellular organisms, multicellular organisms, bacteria, parasites, fungi, protists, algae, larvae, nematodes, worms, and combinations thereof. In various embodiments, the microorganisms are bacteria.

In various embodiments, the portion of the volume of matter of steps i) and iii) is each individually selected from the group consisting of less than 95%, 90%, 85%, 80%, 75%, 70%, 65%, 60%, 55%, 50%, 45%, 40%, 35%, 30%, 25%, 20%, 15%, 10%, 5%, and 1% of total volume of matter.

In various embodiments, an amount of reduction of microorganisms is selected from the group consisting of at least 95%, 90%, 85%, 80%, 75%, 70%, 65%, 60%, 55%, 50%, 45%, 40%, 35%, 30%, 25%, 20%, 15%, 10%, 5%, 1%, to 0.01% reduction of microorganisms in the treated portion of the volume of matter. In various embodiments, the method results in an amount of reduction of microorganisms in a total volume of matter selected from the group consisting of at least 95%, 90%, 85%, 80%, 75%, 70%, 65%, 60%, 55%, 50%, 45%, 40%, 35%, 30%, 25%, 20%, 15%, 10%, 5%, and 1% reduction of microorganisms.

The method can be repeated over a period of time. For example, the period of time may be selected from up to 1 hour, 4 hours, 8 hours, 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years and 5 years. In various embodiments, the method is repeated sequentially for a number of cycles selected from the group consisting of at least 2, 3, 4, 5, 10, 20, 50, 100, 500, 1000, 10,000, 100,000, and 250,000 cycles.

The surface having one or more electrodes can form part or all of a container encompassing the volume of matter. In various embodiments, the surface having one or more electrodes may form part or all of a conduit for transporting the volume of matter. Alternatively, the surface having one or more electrodes forms a surface immersed in the volume of matter. For example, the surface immersed in the volume of matter may be in the form of an insert for a packaged material. In various embodiments, the surface having one or more electrodes is contained in a medical device.

In one aspect, one or more electric fields disclosed herein range from 50 V/cm to 100 kV/cm. In various embodiments, the electric fields are generated with low voltage. For example, the electric fields may be generated by a voltage selected from less than about 10,000V, 1000V, 100V, 10V, 1V, and 0.5V. In one embodiment, the voltage is less than about 10V. The electric fields may be generated with direct current (DC), alternating current (AC), or pulsed current. In various embodiments, the electric fields are pulsed microsecond or nanosecond electric fields.

In various embodiments, the electric fields are substantially non-uniform throughout the volume of matter.

With regard to the exposed microorganisms, in various embodiments, at least a portion of exposed microorganisms undergo electroporation. The electroporation may be selected from the group consisting of reversible electroporation, irreversible electroporation, and combinations thereof.

Surfaces having at least 2 electrodes are encompassed within the disclosure. When two or more electrodes are present, they are separated by a gap. In various embodiments, the gap is between 10 nanometers and 500 microns. For example, the gap may be between 10 nanometers and 50 microns, between 10 nanometers and 10 microns, or between 10 nanometers and 100 nanometers. In various embodiments, the gap comprises an insulator.

In various embodiments of the present disclosure, electrodes are arranged in a co-planar configuration. In various embodiments, at least one electrode comprises a layer of conductive material contacted by a layer of insulating material containing a plurality of gaps. For example, the gaps may have a dimension of less than about 500 microns. In various embodiments, the gaps are spaced at about 100 microns to 100 nanometers apart. In various embodiments, the surface containing the electrodes, and/or the electrodes themselves, are flexible. In various embodiments, the surface containing the electrodes, and/or the electrodes themselves, are not flexible.

In various embodiments, the surface further comprises electrically conductive particles or an electrically conductive polymer or an electrically insulating polymer.

In various embodiments, the methods disclosed herein do not raise a temperature for the volume of matter by more than 50, 40, 30, 20, or 10° C.

Further disclosed are methods for increasing the shelf-life of a perishable composition comprising the step of preparing a perishable composition and performing the methods disclosed herein on the perishable composition.

Further disclosed are methods for affecting cells in a macroscopic volume of matter, where the method comprises i) contacting a volume of matter with a surface having one or more electrodes and exposing a portion of the volume to one or more electric fields that are sufficient to kill at least a portion of exposed cells, ii) moving the exposed portion of the volume with respect to the surface having one or more electrodes, or moving the surface having one or more electrodes with respect to the exposed portion of the volume, and iii) contacting a previously unexposed portion of said volume of matter with a surface having one or more electrodes and exposing the previously unexposed portion of the volume to one or more electric fields that are sufficient to kill at least a portion of exposed cells. In various embodiments, pressure may also be applied to the volume of matter.

### INCORPORATION BY REFERENCE

All publications, patents, and patent applications mentioned in this specification are herein incorporated by reference to the same extent as if each individual publication, patent, or patent application was specifically and individually indicated to be incorporated by reference

### BRIEF DESCRIPTION OF THE DRAWINGS

The novel features of a device of this disclosure are set forth with particularity in the appended claims. A better understanding of the features and advantages of this disclosure will be obtained by reference to the following detailed description that sets forth illustrative embodiments, in which the principles of a device of this disclosure are utilized, and the accompanying drawings.

FIG. 1A is a schematic representation of 2 electrodes and the relationship between electric field, voltage and distance.

FIG. 1B is a schematic representation of co-linear or co-planar electrodes, facing a fluid on a substrate. The figure also shows the corresponding voltage profile of the electrodes.

FIG. 1C is a schematic representation of a singularity device and electric field.

FIG. 2A is a schematic representation of multiple electrodes on surfaces for a container. The electrodes are separated by gaps.

FIG. 2B is a schematic illustration of side and top views of an inter-digitated pattern of electrodes.

FIG. 3A is a schematic representation of a volume of matter in contact with 2 co-planar or co-linear electrodes.

FIG. 3B is a schematic representation of a volume of matter on a surface, in indirect contact with 2 co-planar or co-linear electrodes.

FIG. 3C is a schematic representation of 2 volumes of matter in contact with the sides of an electrode and insulating layers.

FIG. 3D is a schematic representation of a 2 volumes of matter in contact with the sides of an electrode and insulating layers opposite a second electrode.

FIG. 4A is a schematic representation of electrophoresis and dielectrophoresis. The figure on top left is electrophoresis and the other two show dielectrophoresis. Positively charged particles are directed toward a negatively charged electrode and negatively charged particles are directed toward a positively charged electrode.

FIG. 4B is a schematic representation of electrodes, separated by insulating layers, in a ring configuration.

FIG. 4C is a schematic representation of multiple electrodes 490 separated by a gap 495 and further separated by insulating layers 480.

FIG. 4D is a schematic representation of 2 electrodes separated by a gap and insulating layers.

FIG. 5A is a schematic representation of the extent of the portion of the entire matter in which the electric field is effective in treating the matter in the container. In one configuration, the portion of the matter is near the surface of the container. In another configuration, the portion of the matter is in the middle of the container.

FIG. 5B is a schematic representation of the electrodes that generate the electric field to treat a portion of the entire matter in the container. In one configuration, the electrodes that treat the portion of the matter are on the surface of the container. In another configuration, the electrodes are in the middle of the container.

FIG. 5C is a schematic representation of an electric field, generated by 2 co-planar or 2 co-linear electrodes, in a portion of the matter in a conduit. In one configuration, the electrodes are on the surface of the conduit. In another configuration, the electrodes are inserted into the middle of the conduit.

FIG. 5D is a schematic representation of the extent of the electric field that treats a portion of the matter in a conduit. In one configuration the electric field is generated by the surfaces of the conduit. In another configuration the electric field is generated by an insert in the middle of the conduit.

FIG. 6 is a schematic representation of a multi-pulse profile. Low voltage, high frequency pulses designed for dielectrophoresis are followed by high voltage low frequency pulses for irreversible electroporation (IRE). Low voltage, high frequency pulses may follow IRE to remove particles.

FIG. 7A is a schematic representation of a device with a series of ring-like electrodes and insulators. Ring-like metal electrodes (720) are separated by by ring-like insulators (710). Each electrode is attached with wires to a power supply. Leads are shown by 730.

FIG. 7B is a schematic representation of a beaker of water with microorganisms and a device like that in FIG. 7A positioned alongside the beaker.

FIG. 7C is a schematic representation of a device 750 comprising multiple layers of Pyrulax™, fastened with a screw and attached to wires 740 to power the device.

FIG. 7D is a schematic representation of a device comprising a layer of gold 770 with a laser etched gap 760.

FIG. 7E is a photograph of the results of an experiment with a device of FIG. 7D. The left panel shows cells before treatment with the device. The right panel shows dead cells stained dark 780, along the laser etched gap 790, after treatment with the device.

FIG. 7F is a schematic representation of a device comprising inter-digitated gold electrodes on a glass substrate attached to a power supply with aluminum tape.

FIG. 8 is a schematic representation of a beverage container.

FIG. 9A is a schematic representation of an electrode pattern on paper. The same concept of mutilayered structure as that shown for the cardboard could be used with layers of plastic materials instead of paper.

FIG. 9B is a schematic representation of an electrode pattern on non-conductive paper.

FIG. 10A is a schematic representation of a top and cross section review of a rotating shaft attached to a paddle in a container.

FIG. 10B is a schematic representation of a bottle for contact lens solution containing a stir bar with electrodes. The bottle is resting on a magnetic plate unit responsible for rotation of the stir bar.

FIG. 11A is a schematic representation of a contact lens case that contains electrodes.

FIG. 11B is a schematic representation of a contact lens case containing electrodes that are powered when the lids of the case are contacted with the base of the case.

FIG. 12A is a schematic representation of an electric field generating insert that contains electrodes.

FIG. 12B is a schematic representation of electrodes that may be found on any of the surfaces of the insert shown in FIG. 12A.

FIG. 13A is a schematic representation of electrodes found in a serpentine arrangement that are found on the inner surface of a pipe or conduit.

FIG. 13B is a schematic representation of electrodes found in a serpentine arrangement, similar to FIG. 13A that are found on the outer surface of the pipe or conduit.

FIG. 14 is a schematic representation of electrodes on the surface of a roller used to process or extrude products or material. Pressure is also applied to the products as they are moved under the roller.

FIG. 15 is an example of a configuration of a conveyor belt on electric field generating electrodes.

### DETAILED DESCRIPTION OF THE INVENTION

It is an object of the present disclosure to provide compositions, methods, and devices for the reduction of cell content using low voltage, high strength electric fields. Devices according to the disclosure are generally capable of reducing contamination by microorganisms in a portion of a volume of matter using one or more arrangements of electrodes configured to generate high electric fields powered by low voltages for a variety of applications. For example, electrodes are configured on a surface powered by a low voltage to provide one or more high electric fields (LVHEF), strong enough to kill or attenuate all or a portion of microorganisms present in the vicinity of the electric fields. The compositions, methods and devices of the disclosure may be useful in a variety of applications, most notably, sterilization or de-contamination applications involving fluids and solids such as food, beverages, drugs, contact lenses and medical products as well as applications involving packaging, storage or delivery of such fluids, or any application where reduction in biological contamination is desirable. Other notable applications may include the incorporation of compositions, methods and devices of the disclosure in generating surfaces with reduced microbial contamination, with applications ranging from various consumer products to use in various sterile or partially sterile environments and environments prone to microbial growth.

The compositions, methods and devices of this disclosure may be standalone, such as in the example of a container incorporating elements of the disclosure in the walls of the container, for the purposes of reducing microbial contamination in the fluid contents of the container. In other cases, the device may be a component of a larger system such as a separate sterilization device that is added to a container of fluid within a distribution network for that fluid. In other cases, the device may be a component of a larger system such as a surface where a reduction in microbial contamination is desirable. In other cases the device can be a stand-alone insert introduced in a container. In other cases the device can be a pipe through which the treated matter can flow. In other cases the device can be a structure across which the treated matter can flow.

### I. Definitions

The terminology of the present disclosure is for the purpose of describing particular embodiments only and is not intended to be limiting of compositions, methods and devices of this disclosure.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

The term reduction, as used herein with respect to microorganisms, generally refers to any means that results in any decrease in the number of viable microorganisms. In some cases, reduction may result in an amount that is 95%, 90%, 85%, 80%, 75%, 70%, 65%, 60%, 55%, 50%, 45%, 40%, 35%, 30%, 25%, 20%, 15%, 10%, 5%, or 1% of the original amount. In various cases, reduction is indicated by a logarithmic scale, such as a 1-log, 2-log, 3-log, 4-log, 5-log, 6-log, 7-log, 8-log, 9-log, or 10-log reduction. In various embodiments, the reduction is a 3-log reduction. In various embodiments, the reduction is a 5-log reduction. The term reduction may be used interchangeably with other terms such as killing, attenuating, or depleting.

The term volume of matter, as used herein, generally refers to a discrete amount of matter, which may include, but is not limited to, solids, liquids, gases, fluids, mixtures, colloids, gels, aerosols, foams, emulsions, suspensions, and solutions.

The term “sterilization” includes a reduction in microbial content, the amount of which may vary depending on the application as dictated by regulatory agencies.

The term “macroscopic” generally refers to a scale of volume that may be observed by the naked eye. In various embodiments, macroscopic refers to a volume that is at least 1 mm³, 10 mm³, 50 mm³, 75 mm³, 100 mm³, 200 mm³, 300 mm³, 400 mm³, 500 mm³, 600 mm³, 700 mm³, 800 mm³, 900 mm³, 1 cm³, 10 cm³, 50 cm³, 75 cm³, 100 cm³, 200 cm³, 300 cm³, 400 cm³, 500 cm³, 600 cm³, 700 cm³, 800 cm³, 900 cm³, 1 m³, 2 m³, 3 m³, 4 m³, 5 m³, 6 m³, 7 m³, 8 m³, 9 m³, 10 m³, 50 m³, 75 m³, 100 m³, 200 m³, 300 m³, 400 m³, 500 m³, 600 m³, 700 m³ 800 m³, 900 m³, or 1000 m³.

### II. Low Voltage High Electric Fields (LVHEF)

A. Electrodes and Configuration

This disclosure provides for the generation of electric fields strong enough to kill or attenuate microorganisms in a portion of a volume of matter of interest, through reversible electroporation, irreversible electroporation or nanosecond pulses, while requiring relatively low voltages to generate such fields (Low Voltage High Electric Field (LVHEF). These low voltages are low relative to the voltages that would be required to kill or attenuate microorganisms in the entire volume of matter of interest through the same mechanisms of cell death, when applied at once. The volume of matter exposed to the electric fields may be mixed or moved with respect to the unexposed portions of the volume of matter. The mixed or moved volume of matter may be further exposed to electric fields and the process iterated various times. The specific configuration of the electrodes, or how electrodes are arranged with respect to the volume of matter, may be useful to this function.

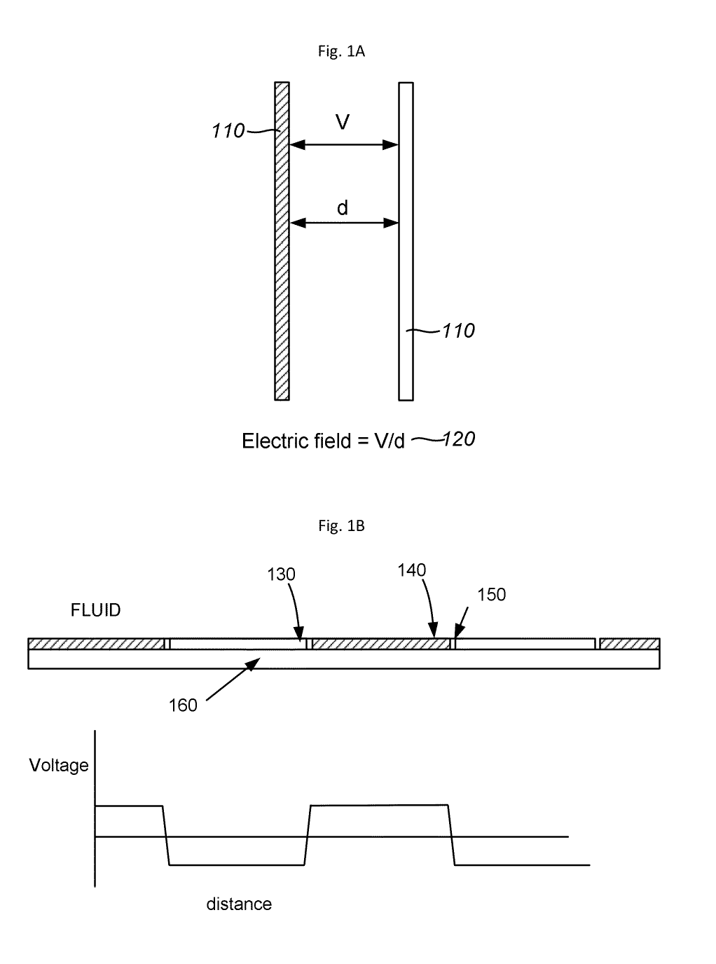

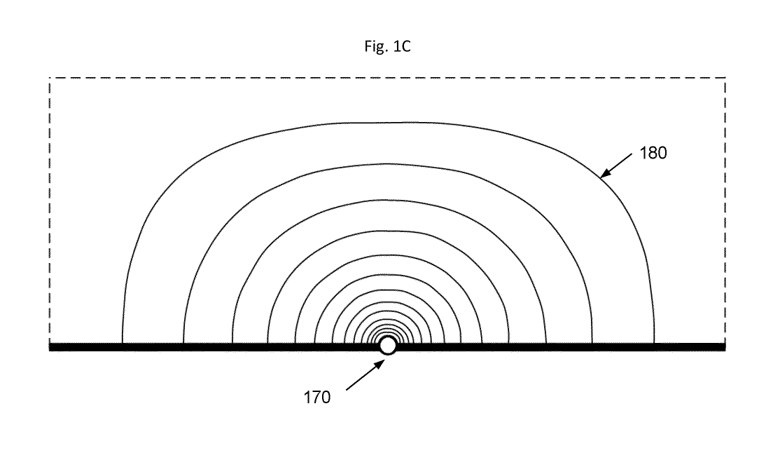

Generally, the strength of an electric field is inversely proportional 120 to the distance between electrodes 110 responsible for generating that field, given a constant voltage as shown in FIG. 1A. Given a constant voltage, a higher electric field may be generated between electrodes that are spaced more closely together. Alternatively, with an increase in distance between electrodes, a higher voltage may also be used to generate stronger electric fields. For example, to generate an electric field of 10,000 V/cm across 2 mm spacing, a voltage difference of 2000 V is needed between the electrodes. A spacing of 0.5 mm may require, for the same field, only 500 V. For 4 mm spacing, 4000 V may be needed. The compositions, methods and devices of the disclosure may utilize this principle, providing for a configuration of electrodes or electrode surfaces that are generally separated by very small distances, producing high fields and requiring low voltages.

As in FIG. 1B, in some instances, electrodes may be arranged such that two or more electrodes, a cathode 130 and anode 140 are exposed in a co-planar or co-linear fashion with a volume of matter. A gap is shown by 150. A substrate is shown by 160. This arrangement is sometimes referred to a singularity device, also shown in FIG. 1C, whereby the distance between the electrodes may be infinitesimally small. (See Gregory D. Troszak and Boris Rubinsky, “A primary current distribution model of a novel micro-electroporation channel configuration,” Biomed. Microdevices 2010 October; 12(5):833-40”; see also PCT/US11/3806, incorporated by reference herein.) A gap is shown by 170, and sample electric field lines are shown by 180. In some cases, a singularity based device may be arranged to allow flexibility with regards to the volume of matter to be exposed to an electric field counter to other electrode arrangements.

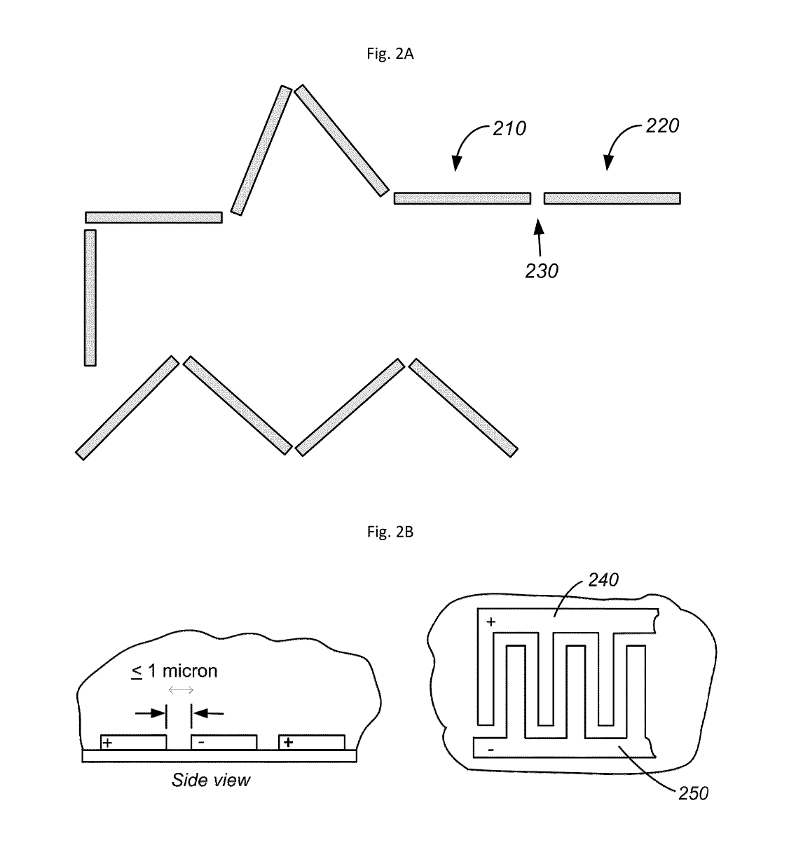

Singularity based devices may comprise a variety of different electrode configurations. As shown in FIG. 2A electrode arrangements include but are not limited to alternating patterns, along the surface of a device, whereby one or more anodes 210 and cathodes 220 may be arranged in a co-linear or co-planar manner, separated by an insulating space or gap 230. In some cases, devices may be found as in FIG. 2B, whereby electrodes 240 and 250 are found in an inter-digitated pattern.

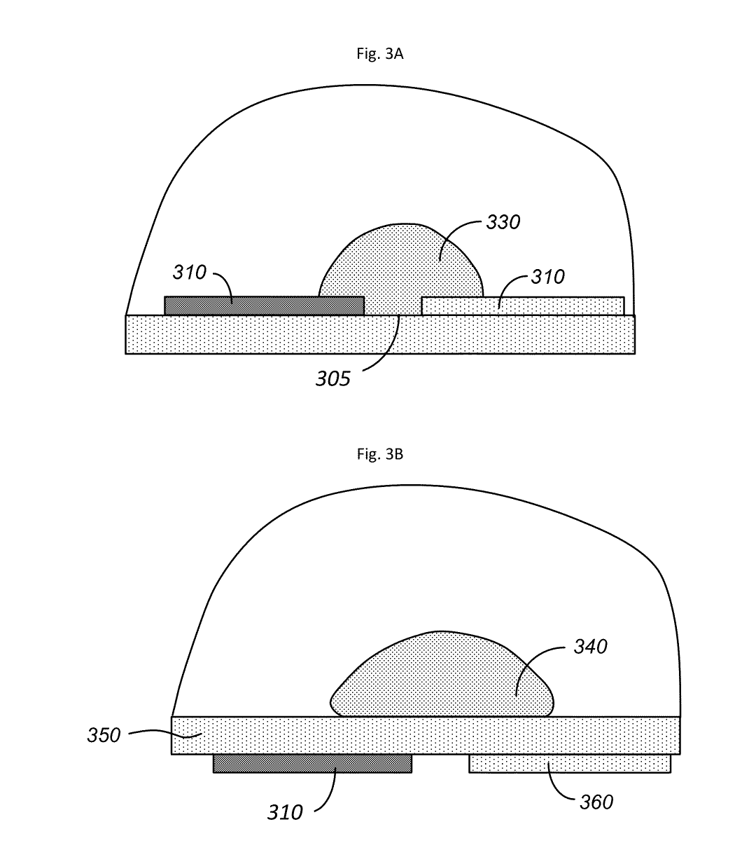

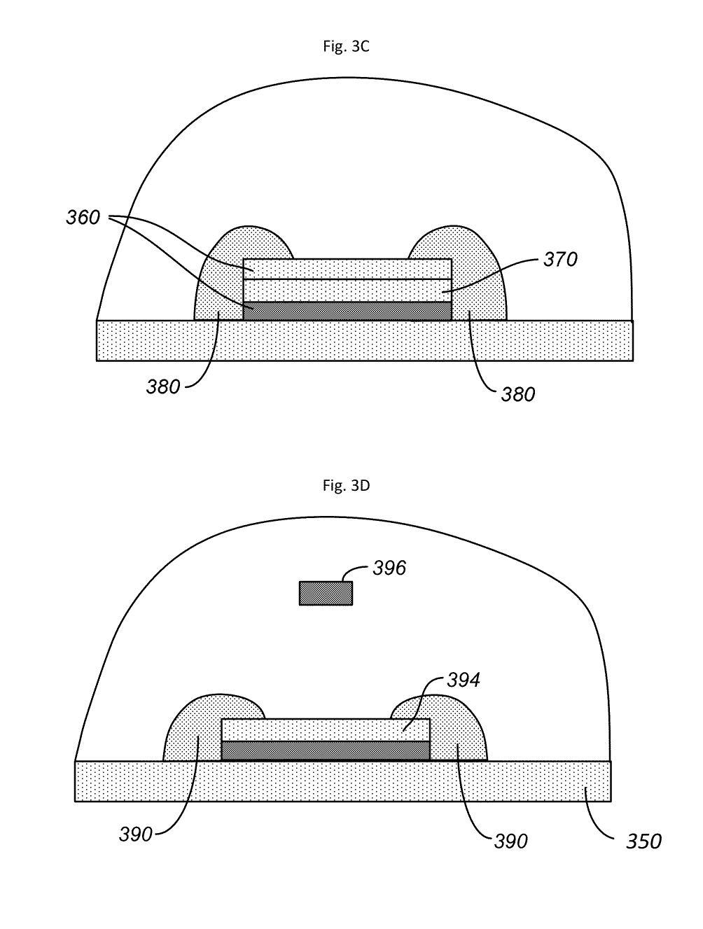

Electrodes may be configured in any arrangement or geometry, whereby a singularity based configuration may be utilized. As in FIG. 3A-D, electrodes may be configured in various arrangements with respect to a volume of matter. In some cases, as in FIG. 3A, a treated part of the volume of matter 330 may directly contact one or more electrodes 310 near spaces, or insulating positions 305 separating the electrodes. In some cases, the treated part of the volume of matter, 340, may not be in contact directly with the electrodes but may contact insulating material 350 which may be in contact with electrodes 310 and 360 as in FIG. 3B. In some cases, electrodes 360 may be arranged in layers, separated by an insulating material 370, whereby the treated part of the volume of matter 380 is in contact with one or more layers as in FIG. 3C. In some instances, as in FIG. 3D, one or more electrodes may not be in contact with one another. In some cases, a treated part of the volume of matter 390 may be in contact with one or more electrodes, or insulating surfaces 394 or layers, while one or more other electrodes 396 may be found in another portion of the device.

This disclosure provides for electrodes which may vary in number. Surfaces may have at least 2, 3, 4, 5, 6, 7, 8, 9, 10, 25, 50, 75, 100, 150, 200, 250, 300, 400, 500, 600, 700, 800, 900, 1000, or 10,000 electrodes. In other cases, the number is less than 3, 4, 5, 6, 7, 8, 9, 10, 25, 50, 75, 100, 150, 200, 250, 300, 400, 500, 600, 700, 800, 900, 1000, or 10,000 electrodes.

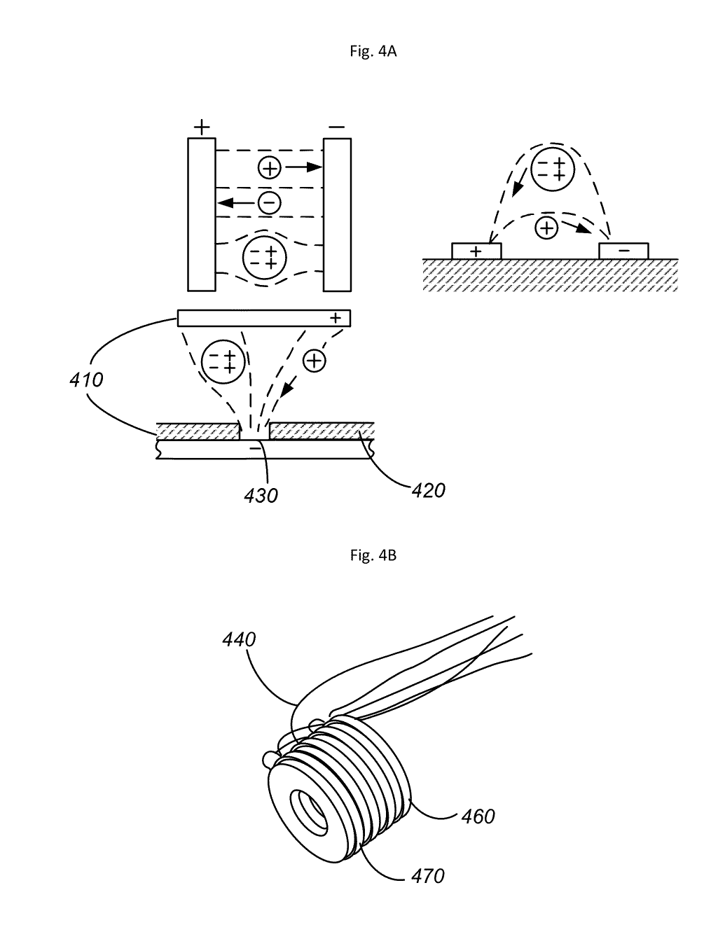

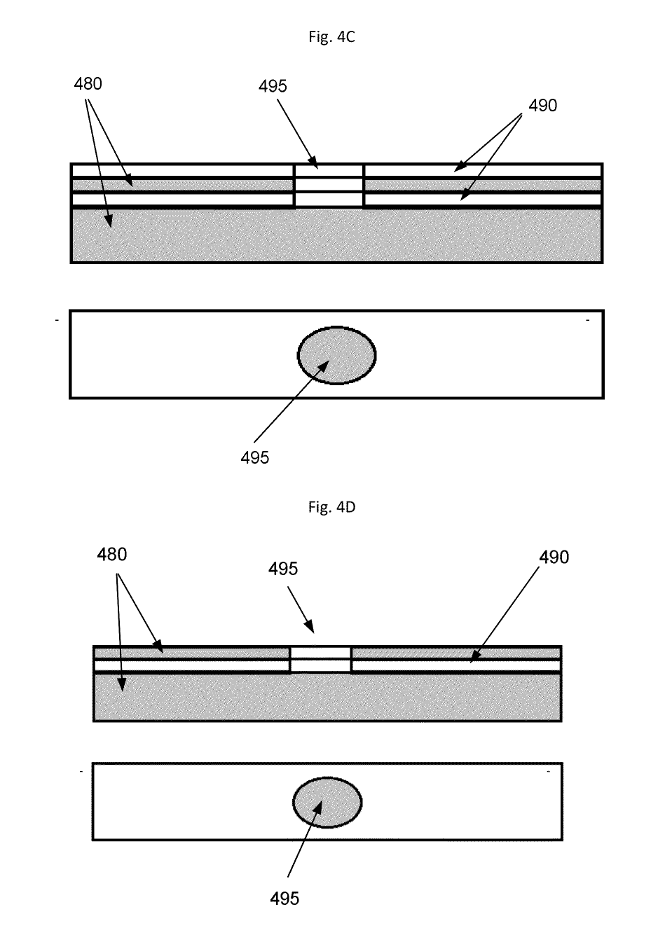

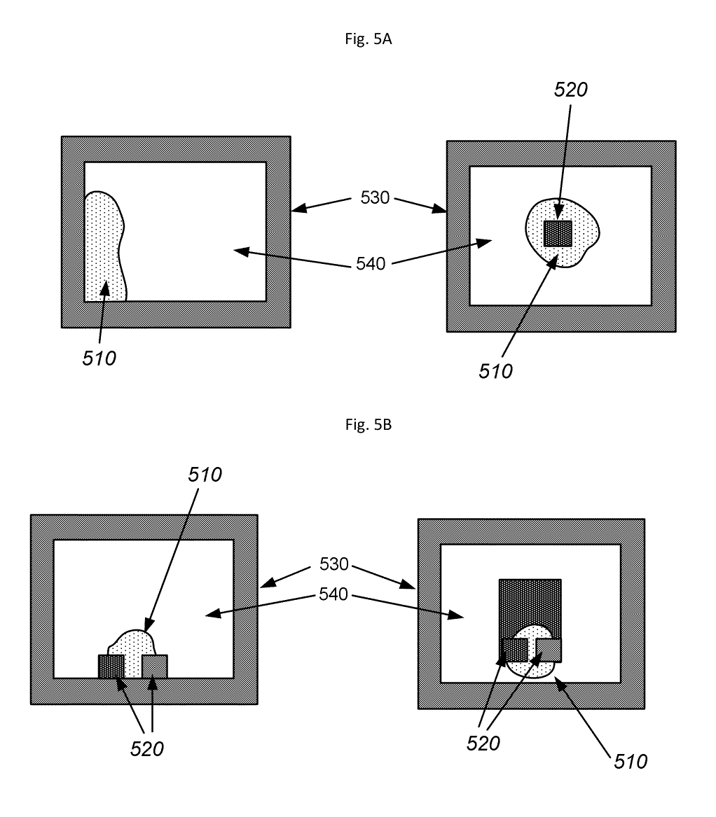

Alternatively, the compositions, methods and devices of the disclosure also provide for other arrangements of electrodes that may not comprise co-planar or co-linear arrayed electrodes, as shown in FIG. 4A-D. In one example, FIG. 4A, electrodes may be juxtaposed 410 with one electrode covered with an insulating material 420 that may be punctuated with a plurality of small gaps, holes or discontinuous spaces 430 in the insulating material. This configuration of electrodes may also provide for electric fields with sufficient strength, focused through the gaps or holes, while requiring relatively low voltages.

Alternatively, the compositions, methods and devices of the disclosure also provide for an insert on which the arrangement of electrodes is in a circular configuration. The electrode may be cylindrical or substantially cylindrical, coiled, or ring-shaped, and separated by insulated structures. See, for example, FIG. 4B. Leads are shown by 440, and metal rings (electrodes) separated by non-metal rings (gaps) are shown by 460 and 470, respectively.

In some cases, although no direct electric contact may occur between the volume of matter and the electrodes, an inductive or a capacitive effect provides for an electric field in at least a portion of the volume of matter. In some cases, an electric field generated is generated with sufficient high strength to kill or attenuate microorganisms through electroporation, but may require low voltage.

The devices of this disclosure provide electrodes that may be spaced by a variety of distances. In some cases, the distances between electrodes is selected from less than 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50 nm, 100 nm, 200 nm, 300 nm, 400 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 50 μm, 100 μm, 200 μm, 300 μm, 400 μm, 500 μm, 1.0 mm, 1.1 mm, 1.2 mm, 1.3 mm, 1.4 mm, 1.5 mm, 1.6 mm, 1.6 mm, 1.7 mm, 1.8 mm, 1.9 mm, 2.0 mm, 3.0 mm, 4.0 mm, 5.0 mm, 6.0 mm, 7.0 mm, 8.0 mm, 9.0 mm, 10 mm, 20 mm, 30 mm, 40 mm, 50 mm, 60 mm, 70 mm, 80 mm, 90 mm, 100 mm, 200 mm, 300 mm, 400 mm, 500 mm, 600 mm, 700 mm, 800 mm, 900 mm, or 1 cm. In other cases, the distances are at least 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50 nm, 100 nm, 200 nm, 300 nm, 400 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 50 μm, 100 μm, 200 μm, 300 μm, 400 μm, 500 μm, 1.0 mm, 1.1 mm, 1.2 mm, 1.3 mm, 1.4 mm, 1.5 mm, 1.6 mm, 1.6 mm, 1.7 mm, 1.8 mm, 1.9 mm, 2.0 mm, 3.0 mm, 4.0 mm, 5.0 mm, 6.0 mm, 7.0 mm, 8.0 mm, 9.0 mm, 10 mm, 20 mm, 30 mm, 40 mm, 50 mm, 60 mm, 70 mm, 80 mm, 90 mm, 100 mm, 200 mm, 300 mm, 400 mm, 500 mm, 600 mm, 700 mm, 800 mm, 900 mm, or 1 cm.

Similarly, a diameter for a hole or gap in insulating material as described herein may be selected from less than 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50 nm, 100 nm, 200 nm, 300 nm, 400 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 50 μm, 100 μm, 200 μm, 300 μm, 400 μm, 500 μm, 1.0 mm, 1.1 mm, 1.2 mm, 1.3 mm, 1.4 mm, 1.5 mm, 1.6 mm, 1.6 mm, 1.7 mm, 1.8 mm, 1.9 mm, 2.0 mm, 3.0 mm, 4.0 mm, 5.0 mm, 6.0 mm, 7.0 mm, 8.0 mm, 9.0 mm, 10 mm, 20 mm, 30 mm, 40 mm, 50 mm, 60 mm, 70 mm, 80 mm, 90 mm, 100 mm, 200 mm, 300 mm, 400 mm, 500 mm, 600 mm, 700 mm, 800 mm, 900 mm, or 1 cm. In other cases, the diameter is at least 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 50 nm, 100 nm, 200 nm, 300 nm, 400 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 50 μm, 100 μm, 200 μm, 300 μm, 400 μm, 500 μm, 1.0 mm, 1.1 mm, 1.2 mm, 1.3 mm, 1.4 mm, 1.5 mm, 1.6 mm, 1.6 mm, 1.7 mm, 1.8 mm, 1.9 mm, 2.0 mm, 3.0 mm, 4.0 mm, 5.0 mm, 6.0 mm, 7.0 mm, 8.0 mm, 9.0 mm, 10 mm, 20 mm, 30 mm, 40 mm, 50 mm, 60 mm, 70 mm, 80 mm, 90 mm, 100 mm, 200 mm, 300 mm, 400 mm, 500 mm, 600 mm, 700 mm, 800 mm, 900 mm, or 1 cm.

The electrode portion of the compositions, methods and devices of the disclosure may be made of any conductive or semi-conductive material. Generally, electrodes may comprise any material that may be sufficient to create an anode or cathode. In some cases, electrodes may include highly conductive and reactive metals, e.g. Cu, Zn, Al etc. In other instances, electrodes may include highly conductive and chemically inert materials e.g. Au, Pt, Ti or inert nonmetallic conductors and insulators, e.g. carbon, graphite. Any material used for an electrode may be coated with an additional conductive polymer or coating.

In some cases, electrodes may be configured and manufactured such that they are flexible. In some cases, electrodes may be manufactured from materials or configured with dimensions that allow the electrode to be bent or manipulated, e.g. thin or fine metal wires, rods or strips. In other cases, electrodes may be configured and manufactured such that they are not flexible. In some cases, electrodes may be manufactured from materials or configured with dimensions whereby electrodes are rigid, e.g. thick ceramic rods.

Additionally, surfaces containing electrodes may be flexible or non flexible. In some cases it may be desirable to configure flexible electrodes on a flexible surface, such as a plastic film or sheet. In some cases, it may be desirable to configure non flexible electrodes on non flexible surfaces, such as on the surface of a rigid container, e.g. bottle, contact lens container, vial.

Electrodes may be configured for any variation of dimensions, e.g. length, width, height etc., size or shape that may be suitable for a desired application, composition, method or device. This disclosure provides for electrodes with any dimensions, sizes or shapes, or any combination thereof.

In various embodiments, the electrodes have a sealant to prevent chemical contamination of the volume of matter in which they are in contact or to prevent chemical contamination of the electrode itself from the volume of matter in which it is in contact. For example, such a sealant may be comprised of any sealant conventionally used in the food, cosmetic, or pharmaceutical arts for packaging materials or for surfaces that come in contact with such materials. In various embodiments, the sealant is physiologically inert. In various embodiments, the sealant reduces or eliminates oxidation of the electrodes. In various embodiments, the sealant does not affect the electric field.

B. Electric Fields, Voltages and Pulses

i. Electric Fields

Generally, configurations of electrodes generate electric fields, which may comprise various strengths. In some cases, electric fields may be strong enough to kill or attenuate microorganisms in the vicinity of the electric field. In some cases, the electric fields may not be of sufficient strength to completely kill or attenuate microorganisms through electroporation. In some cases, multiple electric fields with similar strengths may be suitable. In some cases, multiple electric fields of differing strengths may be suitable. This disclosure provides for one or more similar or differing strength electric fields or any combination thereof.

Compositions, methods and devices of the disclosure generally provide for electric fields strengths which are at least 1 V/cm, 2 V/cm, 3 V/cm, 4 V/cm, 5 V/cm, 6 V/cm, 7 V/cm, 8 V/cm, 9 V/cm, 10 V/cm, 11 V/cm, 12 V/cm, 13 V/cm, 14 V/cm, 15 V/cm, 16 V/cm, 17 V/cm, 18 V/cm, 19 V/cm, 20 V/cm, 50 V/cm, 100 V/cm, 200 V/cm, 300 V/cm, 400 V/cm, 500 V/cm, 1 kV/cm, 2 kV/cm, 3 kV/cm, 4 kV/cm, 5 kV/cm, 6 kV/cm, 7 kV/cm, 8 kV/cm, 9 kV/cm, 10 kV/cm, 11 kV/cm, 12 kV/cm, 13 kV/cm, 14 kV/cm, 15 kV/cm, 16 kV/cm, 17 kV/cm, 18 kV/cm, 19 kV/cm, 20 kV/cm, 50 kV/cm, 100 kV/cm, 200 kV/cm, 300 kV/cm, 400 kV/cm, 500 kV/cm, 750 kV/cm, 1000 kV/cm or 2000 kV/cm. In other cases, the electric field is less than 3 V/cm, 4 V/cm, 5 V/cm, 6 V/cm, 7 V/cm, 8 V/cm, 9 V/cm, 10 V/cm, 11 V/cm, 12 V/cm, 13 V/cm, 14 V/cm, 15 V/cm, 16 V/cm, 17 V/cm, 18 V/cm, 19 V/cm, 20 V/cm, 50 V/cm, 100 V/cm, 200 V/cm, 300 V/cm, 400 V/cm, 500 V/cm, 1 kV/cm, 2 kV/cm, 3 kV/cm, 4 kV/cm, 5 kV/cm, 6 kV/cm, 7 kV/cm, 8 kV/cm, 9 kV/cm, 10 kV/cm, 11 kV/cm, 12 kV/cm, 13 kV/cm, 14 kV/cm, 15 kV/cm, 16 kV/cm, 17 kV/cm, 18 kV/cm, 19 kV/cm, 20 kV/cm, 50 kV/cm, 100 kV/cm, 200 kV/cm, 300 kV/cm, 400 kV/cm, 500 kV/cm, 750 kV/cm, 1000 kV/cm or 2000 kV/cm.

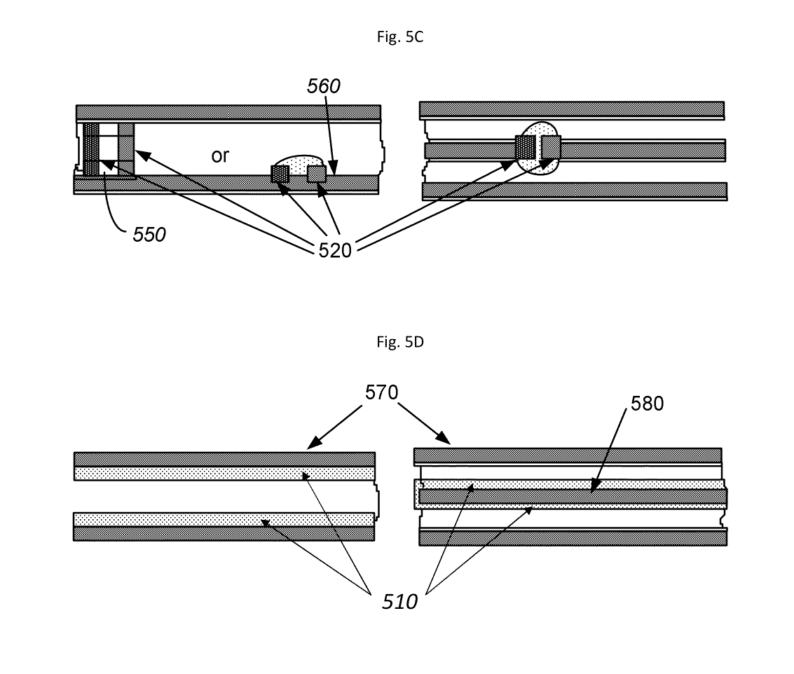

This disclosure provides for electric fields that may be distributed in a volume of matter in a variety of ways as shown in FIG. 5A-D. In some cases, electroporation-type cell reduction inducing electric fields 510 are restricted to a portion of the volume of treated matter as in FIG. 5A, 530 (container) and 540 (volume of matter). In various cases, one or more electric fields 510 may be localized within one site or area as in FIG. 5B, in a volume of matter with an electrode (520). In some cases, one or more electric fields may be localized to multiple sites or areas in a volume of matter as FIG. 5C, 560 (conduit) and 570 (conduit) and gap (550). In some cases one or more electric fields may be localized within in a conduit through which matter flows as in FIG. 5D, including in insert (580). Generally, electric fields localized to a site within a volume of matter may kill or attenuate microorganisms in a specific portion of a volume of matter. In some cases, one or more electric fields may be localized to one or more sites in the volume of matter. Microorganisms may be killed or attenuated by one or more electric fields simultaneously, or sequentially. In some cases, the same microorganisms may be exposed to one or more different electric fields which may be positioned at different sites in the volume of matter. The electric fields can have single polarity or alternating polarity. The electric fields can be produced by DC current or AC current or electric pulses or induction.

ii. Voltages

Generally, electric fields may be generated by voltages comprising various strengths. Voltages may comprise at least 0.1 V, 0.5 V, 1 V, 2 V, 3 V, 4 V, 5 V, 6 V, 7 V, 8 V, 9 V, 10 V, 11 V, 12 V, 13 V, 14 V, 15 V, 16 V, 17 V, 18 V, 19 V, 20 V, 50 V, 100 V, 200 V, 300 V, 400 V, 500 V, or 1 kV. In other cases voltages comprise at most 3 V, 4 V, 5 V, 6 V, 7 V, 8 V, 9 V, 10 V, 11 V, 12 V, 13 V, 14 V, 15 V, 16 V, 17 V, 18 V, 19 V, 20 V, 50 V, 100 V, 200 V, 300 V, 400 V, 500 V, 1 kV or 10 kV.

iii. Electric Pulses

This disclosure provides for electric fields which may be generated as pulses, or fluctuations in various aspects of generated, electric fields such as duration of time, amplitude frequency and profile. In some cases, this disclosure provides no pulse, whereby an electric field is continuously generated.

This disclosure provides for cases in which pulse times may be configured in a variety of ways. In some cases all pulses comprise a similar duration of time. In other cases some pulses times may be similar in duration and some pulses times may be different in their respective durations. This disclosure provides for similar or differing durations of pulses and any combination thereof.

Electric pulses may comprise a variety of duration times. Electric pulse times comprise at least 1 ns, 2 ns, 3 ns, 4 ns, 5 ns, 6 ns, 7 ns, 8 ns, 9 ns, 10 ns, 11 ns, 12 ns, 13 ns, 14 ns, 15 ns, 16 ns, 17 ns, 18 ns, 19 ns, 20 ns, 50 ns, 100 ns, 200 ns, 300 ns, 400 ns, 500 ns, 1 μs, 2 μs, 3 μs, 4 μs, 5 μs, 6 μs, 7 μs, 8 μs, 9 μs, 10 μs, 11 μs, 12 μs, 13 μs, 14 μs, 15 μs, 16 μs, 17 μs, 18 μs, 19 μs, 20 μs, 50 μs, 100 μs, 200 μs, 300 μs, 400 μs, 500 μs, 1.0 ms, 1.1 ms, 1.2 ms, 1.3 ms, 1.4 ms, 1.5 ms, 1.6 ms, 1.6 ms, 1.7 ms, 1.8 ms, 1.9 ms, 2.0 ms, 3.0 ms, 4.0 ms, 5.0 ms, 6.0 ms, 7.0 ms, 8.0 ms, 9.0 ms, 10 ms, 20 ms, 30 ms, 40 ms, 50 ms, 60 ms, 70 ms, 80 ms, 90 ms, 100 ms, 200 ms, 300 ms, 400 ms, 500 ms, 600 ms, 700 ms, 800 ms, 900 ms, or 1 s. In other cases, the electric pulse times comprise less than 3 ns, 4 ns, 5 ns, 6 ns, 7 ns, 8 ns, 9 ns, 10 ns, 11 ns, 12 ns, 13 ns, 14 ns, 15 ns, 16 ns, 17 ns, 18 ns, 19 ns, 20 ns, 50 ns, 100 ns, 200 ns, 300 ns, 400 ns, 500 ns, 1 μs, 2 μs, 3 μs, 4 μs, 5 μs, 6 μs, 7 μs, 8 μs, 9 μs, 10 μs, 11 μs, 12 μs, 13 μs, 14 μs, 15 μs, 16 μs, 17 μs, 18 μs, 19 μs, 20 μs, 50 μs, 100 μs, 200 μs, 300 μs, 400 μs, 500 μs, 1.0 ms, 1.1 ms, 1.2 ms, 1.3 ms, 1.4 ms, 1.5 ms, 1.6 ms, 1.6 ms, 1.7 ms, 1.8 ms, 1.9 ms, 2.0 ms, 3.0 ms, 4.0 ms, 5.0 ms, 6.0 ms, 7.0 ms, 8.0 ms, 9.0 ms, 10 ms, 20 ms, 30 ms, 40 ms, 50 ms, 60 ms, 70 ms, 80 ms, 90 ms, 100 ms, 200 ms, 300 ms, 400 ms, 500 ms, 600 ms, 700 ms, 800 ms, 900 ms, or 1 s.

Further, pulses may be repeated in a variety of patterns and profiles during a cycle. Cycles may comprise at least 1, 2, 3, 4, 5, 10, 20, 50, 100, 500, 1000, 10,000, 100,000, or 250,000 cycles. In other cases, cycles comprise less than 3, 4, 5, 10, 20, 50, 100, 500, 1000, 10,000, 100,000, or 250,000 cycles.

Generally, amplitude of a pulse refers to electric field strength, as described herein. This disclosure provides for instances in which pulse amplitudes are configured in a variety of ways. In some cases all pulse amplitudes may be similar in electric field strength. In other cases, some pulse amplitudes may be different than others in their respective electric field strength. This disclosure provides for similar or differing pulse electric field strength and any combination thereof.

This disclosure provides for cases in which pulse frequency may be configured in a variety of ways. In some cases all pulse frequencies may be similar. In other cases some pulse frequencies may be different and some pulse frequencies may be different. This disclosure provides for similar or differing pulse frequencies and any combination thereof.

Pulse frequencies may be higher than 0.001 Hz, higher than 0.01 Hz, higher than 0.1 Hz, higher than 1 Hz, higher than 100 Hz, higher than 1 kHz, higher than 1 MHz, higher than 100 MHz, or higher than 1 GHz.

Further, the total number of pulses during a cycle may be any number suitable for the applications, compositions, methods or devices of this disclosure. In some cases, the number of pulses may comprise at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, or 20000. In other cases, the number of pulses during a cycle may comprise less than 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 15000, or 20000. The electric pulses can be single polarity or alternating polarity.

In various embodiments, current has a wavelength selected from less than10 minutes, 10⁻² s, 10⁻³ s, 10⁻⁴ s, 10⁻⁵ s, 10⁻⁶ s, 10⁻⁷ s, 10⁻⁸ s, and 10⁻⁹ s.

C. Power and Control

This disclosure provides electrodes that may be powered in a variety of ways. In some cases, electrodes may not be attached to an external power supply, whereby the electrodes themselves may generate an electric field through a difference in electrochemical potential between anode and cathode electrodes. For example, if electrodes are chosen, such that one comprises Cu and one comprises Zn, the difference in potential between the electrodes may be sufficient to generate a desired electric field through an electrolytic reaction without a power from an external source.

Electrical current can be DC or AC or pulsed current. In some cases, electrodes may be attached to an external power supply, driven by either alternating current (AC), direct current (DC) or a combination thereof. In some cases AC or a pulsed current may be passed to an electrode one or more times.

This disclosure provides for a variety of sources that may act to power electrodes to generate electric fields. Sources may include but are not limited to batteries, electric wall socket, a mechanical energy driven electrical power supply or a photovoltaic cell. This disclosure provides for any power sources, or combination thereof, suitable to aid in generation of electric fields suitable for any desired application, composition, method or device.

Additionally, electrodes may be connected by electrical cables to a pulse modulation unit, also referred to herein as a pulsing unit or pulser. The pulse modulation unit may contain the electrical components, i.e. capacitors, waveform generators, AC to DC transformers, etc., for generating the low voltage electrical pulses, by applying a defined DC or AC voltage to the electrodes, and generating one or more pulses with desired parameters as described herein. Pulse modulation units are commercially available. The pulse modulation unit may be proximate or remote from the electrodes.

Further, the process may be controlled by a central processing unit (CPU), which may either be a component of the pulse modulation unit or be separate therefrom. The CPU may be programmed to set operating limits for all control parameters. The principle control parameters may include pulse frequency, pulse and level of applied voltage.

### III. Cells

Generally, a volume of matter to be treated contains cells. In various embodiments, the cells are one or more microorganisms. In some cases, microorganisms may comprise any microscopic living entity. In some cases, microorganisms in a volume of matter may be found as undesirable contamination. Microorganisms may include but are not limited to unicellular organisms or multicellular organisms, bacteria, parasites, fungi, protists, algae, larvae, nematodes, worms, and any combination thereof. In some cases, microorganisms include viruses. In various embodiments, the microorganisms are food borne pathogens. Microorganisms may include pathogenic bacteria including but not limited to Bacillus anthracis, Bordetella pertussis, Borrelia burgdorferi, Brucella abortus, Brucella canis, Brucella melitensis, Brucella suis, Campylobacter jejuni, Chlamydia pneumoniae, Chlamydia psittaci, Chlamydia trachomatis, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureusa, Staphylococcus epidermidis, Staphylococcus saprophyticus, reptococcus agalactiae, Streptococcus pneumoniae, Streptococcus pyogenes, Treponema pallidum, Vibrio cholerae, Yersinia pestis. In various embodiments, microorganisms are selected from the group consisting of E. coli, Salmonella, Clostridium perfringens, Campylobacter spp., Staphylococcus aureus, MRSA, Toxoplasma gondii, Listeria monocytogenes, Bacillus, Shigella spp., Streptococcus, Vibrio, Yersinia, Brucella, Cryptosporidia, and Giardia.

Further, killing or attenuation of microorganisms may comprise a variety of different mechanisms. Generally, reduction, killing or attenuation may comprise any means that results in the death, or inability to reproduce of a cell or microorganism.

In some cases, killing may be occur through irreversible electroporation, whereby electric fields generate pores in the cell membranes of microorganisms, whereby the pores are unable to close. Inner contents of the cells may pass through the membrane, which may kill the cell. In other cases, electric fields may cause irreversible damage to vital cellular structures and features, such as organelles, chromosomes, vesicles, mitochondria, chloroplasts, ribosomes, the nucleus, DNA, RNA and the like. In some cases, the cells or microorganisms may be killed through nanosecond electroporation, while the outer membrane or cell wall remains intact.

In other cases, electric fields may promote other mechanisms for killing microorganisms associated with electroporation such as osmotic shock or changes in the intracellular composition of the cell.

### IV. Volume of Matter

This disclosure provides for a volume of matter, as described herein, that may comprise numerous embodiments. In some cases, a volume of matter may contain ions, charged particles, or charge carriers. In some cases, a volume of matter may not contain any these species. In some cases, a volume of matter may contain any combination thereof. In some embodiments, the volume of matter is a macroscopic volume of matter.

Generally, a volume of matter may include any suitable type of matter. In some cases, matter may include, but is not limited to, liquids such as water, saltwater, saline, brackish water, wastewater, sewage, foods, beverages, milk, juices, mayonnaise, drugs, vaccines, medical products, pharmaceutical products, biotechnology products and cosmetics. In some instances for example, a volume of matter may comprise injectable drugs, e.g. insulin, vaccines, or recombinant drug therapies contained in solution. In some instances, the volume of matter may contain products of compounding pharmacies, large or small molecule cancer therapeutics, insulin, multi-dose vaccines, and the like.

In some cases, a volume of matter may be solid or semi-solid material. In some instances for example, a volume of matter may comprise solid food, meat, or paper pulp.

Further, electric fields may be configured such that the characteristics of the volume of matter may be altered or not altered. In some cases, electric fields may be configured to kill or attenuate microorganism such that the volume of matter is not substantially changed or altered, e.g. physically or chemically. Physical or chemical alterations may include but are not limited to the generation of chemical byproducts, free radicals, ionizing radiation, osmotic shock, heating, turbidity and the like. In some cases, electric fields may be configured such that the temperature of the volume of matter may comprise at least an increase or decrease of 1, 2, 3, 4, 5, 6, 7, 8, 9, 10, 20, 30, 40, or 50° C. In other cases, the temperature of the volume of matter may comprise at most an increase or decrease of 3, 4, 5, 6, 7, 8, 9, 10, 20, 30, 40, or 50° C. In other cases, the volume of matter may be altered after the application of one or more electric fields through chemical or physical means as described herein. In some cases, the taste of the matter is not detectably altered when subjected to human taste tests. In some cases the medical properties of the matter or the medical function of the matter is not changed when subjected to medical applications.

A. Flow

Compositions, methods and devices of this disclosure additionally provide for general mechanism for facilitating exposure of cells or microorganisms with one or more electric fields. Generally, electric fields are configured such that a portion of a volume of matter may be exposed to an electroporation type induced cell killing (including irreversible electroporation, reversible electroporation, nanosecond pulses) electric field at a particular time. In some cases, this may result in the killing or attenuation of a portion of microorganisms in a portion of the volume of matter. In some cases, killing or attenuating microorganisms throughout the entirety of the volume of matter may be desirable.

In some cases, this may be achieved through mechanisms that may promote flow or mixing within the volume of matter. In some cases, electrodes may be fixed such that electric fields may not move relative to the device while the microorganisms may freely move in the volume of matter. Various mechanisms, including but not limited to mechanical agitation, stirring or shaking, convection, bulk flow, forced convection, natural convection (e.g. thermal induced), osmosis, diffusion, and turbulent flow, may increase flow in the volume of matter. This may increase the number of microorganisms that may be exposed to an electric field. In some cases, this may also increase the frequency with which a microorganism may be exposed to an electric field. In some cases this may result in substantial reduction in microorganisms in the volume of matter. An example may include but is not limited to a container comprising electrodes fixed in walls of the container, maybe shaken or stirred to facilitate flow within the contents of the containers. Another example may include but is not limited to a container comprising electrodes in an insert in the container, and the container maybe shaken or stirred to facilitate flow within the contents of the containers. Microorganisms may be present in the contents and may be exposed to one or more electric fields in one or more walls of the container, thus killing them.

Further, in some cases, electrodes may be configured such that they are mobile. In some cases electric fields may be configured to be exposed to different portions of the volume of matter. Mechanisms of flow, such as described herein, may also increase the number of microorganisms that may be exposed to an electric field. In some cases, this may also increase the frequency with which a microorganism may be exposed to an electric field. In some cases this may result in substantial reduction in microorganisms in the volume of matter. An example may include but is not limited to a stir shaft attached to a propeller or paddle comprising electrodes that may be inserted into a container. The shaft may be rotated, creating mechanical agitation of the liquid and facilitating flow within the contents of the containers. Microorganisms may be present in the contents of the container and may be exposed to one or more electric fields on the stir stick during its movement through the container.

B. Movement

In some cases, a portion of a volume of matter may be exposed to one or more electroporation type induced cell-killing electric fields at a particular time through mechanisms that may promote movement of the volume of matter. In some cases, the volume of matter may be fixed such that electrodes and electric fields may move relative to the volume of matter. Movement of either a volume of matter or electrodes relative to the volume of matter, or a combination thereof, may include but are not limited to sliding, pushing, pulling, extruding, rolling, pumping, grinding, or the like. In some cases, movement of a volume of matter may apply to materials which may include solids, semi-solids, gels, or the like. Movement of the volume of matter or electrodes or electrodes relative to the volume of matter may increase the number of microorganisms that may be exposed to an electric field. In some cases, this may also increase the frequency with which a microorganism may be exposed to an electric field. In some cases this may result in substantial reduction in microorganisms in the volume of matter. An example may include but is not limited to a rolling cylinder extruder covered in electrodes, whereby an extruder configured with LVHEF electrodes rolls out pulp or semi-solid material, e.g. paper, plastic, and cardboard, onto a conveyor belt.

C. Electrophoresis and Dielectrophoresis

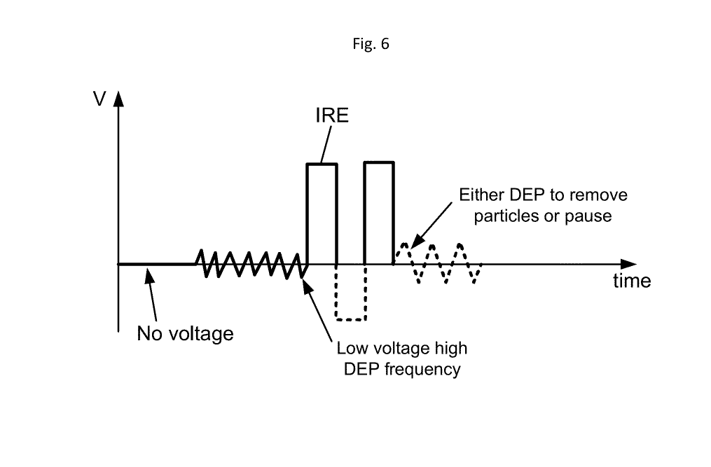

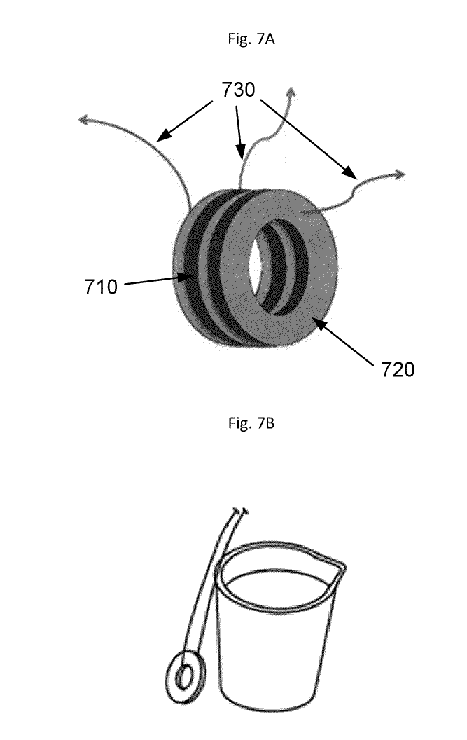

In some cases, compositions, methods and devices of this disclosure additionally provide for general mechanisms for facilitating exposure of microorganisms with one or more electric fields involving dielectrophoresis (DEP) or electrophoresis (EP). DEP or EP generally involves the use of affecting the motions of charged particles in fluids using electric fields. Generally, electric fields exposed to microorganisms may be manipulated, whereby the field may be used to attract or aggregate microorganisms to a vicinity near the electrodes generating the field before supplying enough energy to kill or attenuate the microorganism. The vicinity most near the electrodes may provide an area where electric field strength is stronger than other areas which may increase the likelihood of killing or attenuating microorganisms. An example may include but is not limited to manipulation of electric field amplitudes and pulse times to draw a microorganism, e.g. bacteria, closer to a set of electrodes via DEP or EP. In this example, microorganisms such as bacteria may exhibit a natural charge. Electric fields may first be applied with longer duration, but lower strength. This configuration may facilitate electrostatic attraction of the microorganism closer to the electrodes. After a certain time, the electric fields may be pulsed, such that amplitude or strength is increased for one or more shorter periods of time. Higher amplitude pulses may be sufficient to kill microorganisms. Pulse profiles useful for DEP or EP for attracting microorganisms, followed by pulses useful in irreversible electroporation or killing of microorganisms is shown as an example in FIG. 6.

In some cases, the electrodes generating electric fields to kill or attenuate microorganisms may be the same as the electrodes generating electric fields used for DEP or EP. In other cases, two or more different sets of electrodes may be configured for either DEP or EP or killing or attenuating microorganisms.

In some cases, microorganisms may be attracted towards a cathode via DEP or EP. In other cases, microorganisms may be attracted toward an anode via DEP or EP.

In other cases, one or more cycles of DEP or EP followed by killing or attenuation of microorganisms may be repeated. Any suitable repetition or configuration of DEP and EP steps, alone or in combination may be used for any application, composition, method or device of this disclosure.

In some cases, DEP or EP may not be possible, if a microorganism does not naturally possess a charge, which may be important in electrostatic attraction. In some cases, a chemical additive, or compound, may be added to the volume of matter. This additive may contact microorganisms providing an overall charge on the microorganism. In some cases this may give microorganisms sufficient charge to be affected by DEP or EP.

In some cases, additives may include, but are not limited to resins, polyelectrolytes, salts, ions, and charged particles.

### V. Applications

A. Terminal Sterilization

There are generally two basic methods storage of sterile materials: production for sterile products using terminal sterilization and production for sterile products using aseptic methodologies.

The compositions, methods and devices of this disclosure may be used for terminal sterilization. Generally, this methodology involves filling and sealing product containers in high quality, environments that may be sterile or substantially sterile. The product or container may be subsequently sterilized using any number of techniques, including but not limited to application of heat, irradiation or high pressure. Products and containers are filled and sealed in high quality environments so that accumulation of microbial and particulate content may be minimized in the containers.

B. Aseptic Sterilization

The compositions, methods and devices of this disclosure may be used for aseptic sterilization. Generally, this methodology involves sterilization of a product, container and closure before each is brought together, e.g., filling of the container with product. With this methodology, there may be no separate sterilization step of the product in the container after filling.

C. Terminal Sterilization with Low Voltage High Electric Field (LVHEF) Containers

This disclosure provides for high electric field (LVHEF) containers with the method and the devices of this invention, which may be used for terminal sterilization of products. Generally, low voltage high electric field devices may be configured for any type of container that may require the reduction of microorganisms. Configurations may vary, based on the size, shape and use of the container. For example, for certain containers, electrodes may be embedded in the walls or surfaces of the container. In some instances, electrodes may be powered by an external power source. In other cases, electrodes may be self-powered via differences in electrochemical potential of the electrodes themselves. In some cases, LVHEF containers may be used for storage of contents of the container. In other cases, LVHEF containers may be used for other purposes other than storage of contents in the container.

In another aspect, this disclosure provides for containers which may be sealed before or after a volume of matter is exposed to one or more electric fields. Any suitable sealant material may be used including, but not limited to, TEFLON, rubber, tape, polyeurathane, silicone or acrylics. In some instances, sealants may prevent additional microbial contamination from entering a LVHEF container. In some instances, sealants may prevent oxidation, leakage, damage or spoilage of the contents of the container, which may include the electrodes or the volume of matter.

As provided by this disclosure, electrodes may be embedded or added to any type of container material, including but not limited to cardboard, metal, plastics, polymers, glass, paper, and the like. Any suitable methods may be used for the production, manufacturing or process of electrodes in terminal LVHEF containers.

Generally, LVHEF containers for terminal sterilization may also incorporate various designs for promoting flow in the container. For example, mechanisms for promoting flow in container may include but are not limited to elements added to the container such as a stir shaft attached to a propeller or paddle, a stir bar, a rotating element, shaking, vibrating, or other means of mechanical agitation.

LVHEF containers for terminal sterilization may be used for a variety of purposes that may require reduction of microorganisms. For example, LVHEF containers may be used for products which include but are not limited to food, beverages, milk, juice, mayonnaise, medical products, drugs, water, wastewater, cleaning solutions, cosmetics, pharmaceuticals, contact lens solutions, and the like.

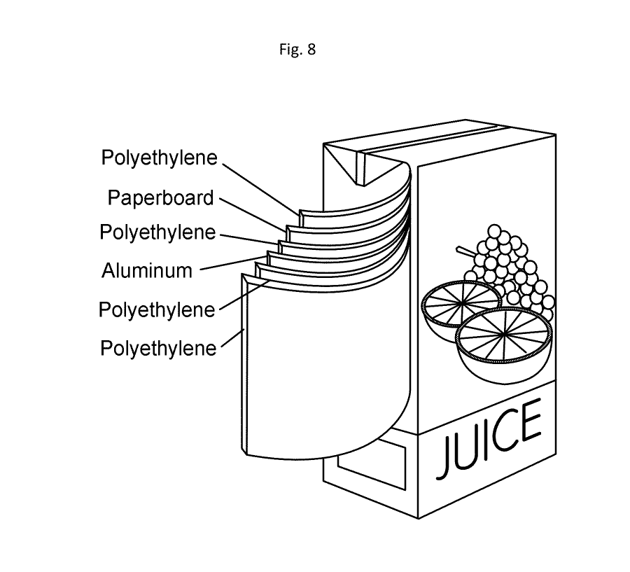

For example, a container may comprise a vial containing glass surfaces, such as used to store pharmaceutical products, e.g. vaccines, injectable drugs, insulin etc. In another example, a container may comprise a beverage carton comprising multiple layers of cardboard, paper, plastic and metal as shown in FIG. 8.

Further, a container may comprise any size, shape or form suitable for the contents of the container. A container may include but is not limited to a bag, beaker, bin, bottle, bowl, box, bucket, can, canister, canteen, capsule, carafe, carton, cask, casket, chamber, cistern, cradle, crate, crock, flask, humidor, hutch, jar, jug, kettle, magnum, package, packet, pail, pod, pot, pottery, pouch, receptacle, sac, sack, storage, tank, tub, vase, vat, vessel, or vial.

Further, this disclosure provides for LVHEF containers which may be used for the reduction of microorganism for other materials which may be placed in container. For example, a LVHEF container may be configured to reduce microorganisms in a liquid which further comprise other objects or contents, including but not limited to cosmetic products, medical products, e.g. surgical tools, contact lenses, hygiene products, e.g. toothbrush, cleaning products, e.g. sponges, brushes, and the like.

D. Aseptic Sterilization with LVHEF Containers

Additionally, this disclosure provides for LVHEF containers which may be used for aseptic sterilization of products. Generally, low voltage high electric field devices may be configured for any type of container that may require the reduction of microorganisms in an aseptic packaging process. Configurations may vary, based on the size, shape and use of the container and for the product being sterilized. For example, for certain containers, electrodes may be embedded in the walls or surfaces of the container. In other cases the electrodes may be on an insert. In other cases, electrodes may be configured as a coil. In some instances, electrodes may be powered by an external power source. In other cases, electrodes may be self-powered via differences in electrical potential of the electrodes themselves. The contents of the container, after microorganisms may have been reduced or eliminated, may be further packaged or combined with other sterile products.

Another example may include but is not limited to LVHEF electrodes incorporated in other distributions elements for aseptic processing. Distribution elements may include but are not limited to channels, conduits, nozzles, pipes, tubes or conveyors, whereby volumes of matter, in transit from one area to another, may be exposed to electric fields. In other cases electrodes may be exposed to fluid in the path of the flow, e.g. of fluid flow through a pipe or conduit. LVHEF electrodes may be configured in a variety of ways for various distribution elements. In one example of a pipe, a serpentine arrangement of electrodes may be configured on the inside wall of the pipe to provide reduction of microorganisms in any matter that flows through the pipe.

Generally, conduits, pipes, channels and tubes may comprise a diameter greater than 0.6 μm, 1.0 μm, 10 μm, 50 μm, 75 μm 100 μm, 200 μm, 300 μm, 400 μm, 500 μm, 600 μm, 700 μm, 800 μm, 900 μm, 1 mm, 10 mm, 50 mm, 75 mm, 100 mm, 200 mm, 300 mm, 400 mm, 500 mm, 600 mm, 700 mm, 800 mm, 900 mm, 1 cm, 10 cm, 50 cm, 75 cm, 100 cm, 200 cm, 300 cm, 400 cm, 500 cm, 600 cm, 700 cm, 800 cm, 900 cm, 1 m, 2 m, 3 m, 4 m, 5 m, 6 m, 7 m, 8 m, 9 m or 10 m. In other cases, the diameter may comprise less than 1.0 μm, 10 μm, 100 μm, 200 μm, 300 μm, 400 μm, 500 μm, 600 μm, 700 μm, 800 μm, 900 μm, 1 mm, 10 mm, 100 mm, 200 mm, 300 mm, 400 mm, 500 mm, 600 mm, 700 mm, 800 mm, 900 mm, 1 cm, 10 cm, 100 cm, 200 cm, 300 cm, 400 cm, 500 cm, 600 cm, 700 cm, 800 cm, 900 cm, 1 m, 2 m, 3 m, 4 m, 5 m, 6 m, 7 m, 8 m, 9 m or 10 m.

As provided by this disclosure, electrodes may be embedded or added to any type of container material, including but not limited to cardboard, metal, plastics, polymers, glass, paper, and the like. Any suitable methods may be used for the production, manufacturing or processing of electrodes in aseptic processing LVHEF containers.

Generally, LVHEF containers for aseptic sterilization may also incorporate various designs for promoting flow in the container. For example, mechanisms for promoting flow in container may include but are not limited to elements added to the container such as a stir shaft attached to a propeller or paddle, a stir bar, or other rotating elements, shaking or other means of mechanical agitation.

LVHEF containers for aseptic sterilization may be used for a variety of purposes that may require reduction of microorganisms. For example, LVHEF containers may be used for the packaging and packaging of products which include but are not limited to food, beverages, milk, juice, medical products, cosmetics, drugs, wastewater and the like.

E. Terminal Sterilization with LVHEF Surfaces

Alternatively, this disclosure also provides for LVHEF surfaces which may be used for terminal sterilization of products. Generally, low voltage high electric field devices may be configured for any type of surface which may be contacted by a volume of matter that may require the reduction of microorganisms. Configurations may vary, based on the size, shape and use of the surface. For example, for certain surfaces, electrodes may be embedded in the surface or applied on top of the surface. In some instances, electrodes may be powered by an external power source. In other cases, electrodes may be self-powered via differences in electrical potential of the electrodes themselves. In some instances the power supply may be within the container or part of the surface, such as a battery

In some instances, a LVHEF surface for terminal sterilization may be added to a container or package that may contain microorganisms. Further, a LVHEF surface may be added to a container for prevention of microbial growth. In some cases LVHEF surfaces may be an insert or other mobile elements. In some cases the insert or mobile elements may also facilitate flow surrounding the LVHEF surface, with similar mechanisms as described herein. In various designs, the insert floats or is not attached to the container. In various designs, the insert is attached to the container.

In some instances, a LVHEF surface may be used for sterilization in an environment that may either require reduction of microorganisms or that may be prone to microbial growth. In some instances, this may be a clean hood or clean surface, whereby LVHEF electrodes are embedded or applied to one or more surfaces to maintain a sterile or substantially sterile environment. In other cases, LVHEF electrodes may be applied to open areas or environments, such areas around a sink or faucet or bathroom fixture. In other cases, LVHEF electrodes may be applied to any consumer product that may be prone to microbial growth or contamination. In other cases, LVHEF electrodes may be applied to medical products which may include but are not limited to medical devices such as catheter, or a prenatal incubator.

F. Aseptic Sterilization with LVHEF Surfaces

Alternatively, this disclosure also provides for LVHEF surfaces which may be used for aseptic sterilization of products. Generally, low voltage high electric field devices may be configured for any type of surface which may be contacted by a volume of matter that may require the reduction of microorganisms. Configurations may vary, based on the size, shape and use of the surface. For example, for certain surfaces, electrodes may be embedded in the surface or applied on top of the surface. In some instances, electrodes may be powered by an external power source. In other cases, electrodes may be self-powered via differences in electrochemical potential of the electrodes themselves.

In some instances, a LVHEF surface for aseptic sterilization may be added to devices used in traditional processing or packaging processes. Further, a LVHEF surface may be added to a container for prevention of microbial growth. In some cases LVHEF surfaces may be an insert or other mobile elements. In some cases the insert or mobile elements may also facilitate flow surrounding the LVHEF surface, with similar mechanisms as described herein. In various designs, the insert floats or is not attached to the container. In various designs, the insert is attached to the container.

In some instances, a LVHEF surface may be used for sterilization in an environment that may either require reduction of microorganisms or that may be prone to microbial growth. In some instances, this may be a roller or extruder, whereby LVHEF electrodes are embedded or applied to one or more rolling or moving surfaces to maintain a sterile or substantially sterile environment during packaging. In some instances, LVHEF electrodes may be used in combination with other processes which may contribute to reduction of cells or microorganisms. In some cases, addition of pressure to a volume of matter while exposed to electric fields generated by LVHEF electrodes may be used.

### EXAMPLES

### Example 1

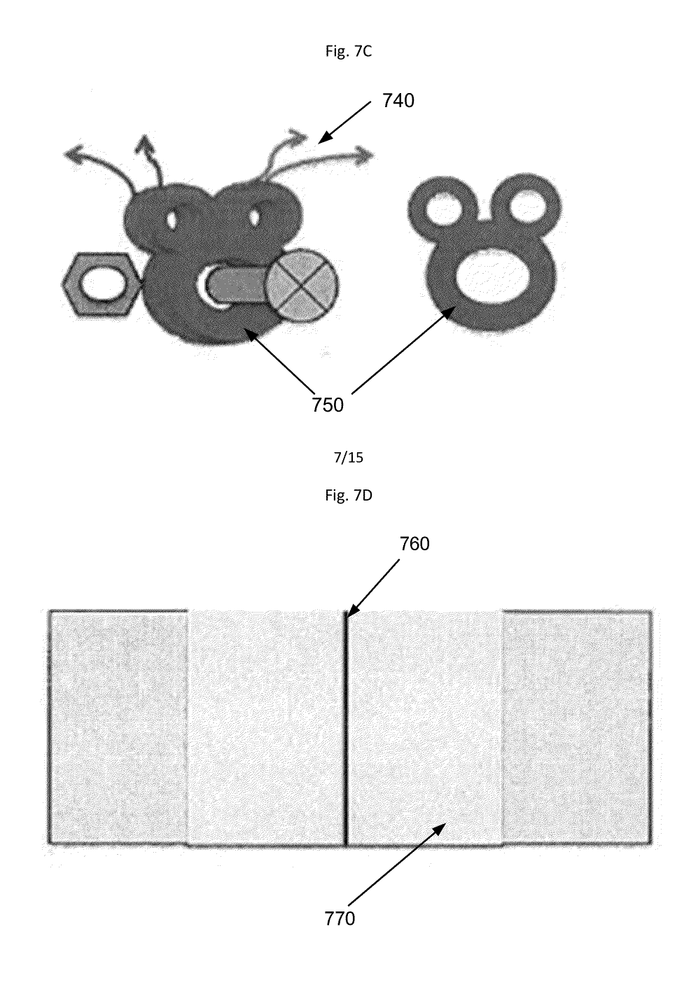

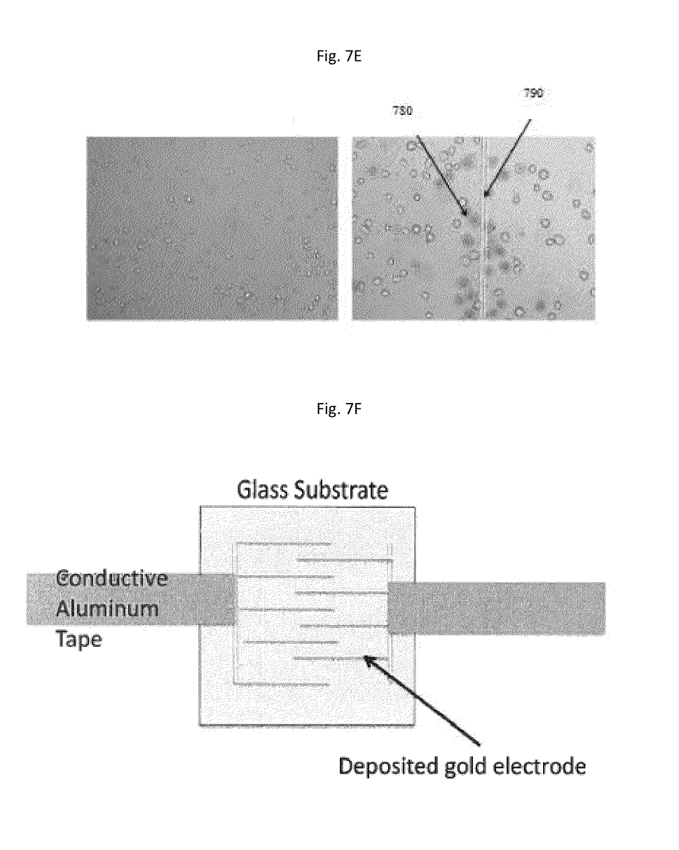

A cylindrical LVHEF device was manufactured from zinc covered stainless steel rings and plastic rings. Three metal rings 720, measuring 0.078 inches in width and 0.8 inches in diameter were bound with glue with two plastic rings 710, in an alternating fashion as shown in FIG. 7A. Wires were attached to each metal ringand further attached to positive and negative outputs 730 on an electroporation pulse generator, Harvard Apparatus BTX.

Efficacy of the device was tested in the presence of baker's yeast. The device was configured to deliver 99 pulses, with pulse lengths of 100 μs, 500V and 4 Hz. 2 pellets of standard baker's yeast (Saccharomyces cerevisiae) were dissolved in 100 ml sterile water and divided into two 50 ml aliquots. One sample was treated with the device and one untreated, serving as a control. Each aliquot was subjected to circulation via stirring rods and covered in parafilm to prevent outside contamination. After 6 hours the treated sample showed visible change in turbidity in the dissolved yeast.

### Example 2

A device as shown in FIG. 7B, was constructed from Pryulax©, a copper coated polyimide material manufactured by Dupont. The copper conductive layer component of the material was selected as 12 μm in thickness and the polyimide component as 9 μm in thickness. A shape, comprising three hollow circles, was used to stamp out 40 layers from the Pryulax material. The dimensions of each layer roughly approximated 1 inch by 1 inch. The layers were assembled by inserting a nylon screw and nut through one of the hollow circles. The screw and nut were tightened to limit distances between individual Pryulax layers. Positive and negative leads were attached to the device and attached to their respective outputs on the Harvard Apparatus BTX device.

Efficacy of the device was tested in the presence of E. coli. The device was configured to deliver 99 pulses, with pulse lengths of 100 μs, 5V and 4 Hz. The device was initiated every 10 minutes over a 5 hour period. 2 ml of E. coli, cultured to stationary phase in LB broth, was added to 100 ml sterile water and divided into two 50 ml aliquots: one treated with the device and one untreated as a control. The device was inserted in one beaker. During the LV HEF procedure, each beaker was subjected to circulation via stirring rods and covered in parafilm to prevent outside contamination. The treated sample showed a 3.5 log reduction in E. coli as compared to the untreated control. Conventional pulsed electric fields treatment of the same volume as that treated here with the LVHEF method would have required voltages of 10,000 V, in comparison to the 500V required by the LVHEF method with mixing.

### Example 3

A LVHEF device was constructed from gold electrodes deposited on a glass cover-slip substrate. Gold was deposited as a 500 nm layer on the glass as shown in FIG. 7D. A laser was used to divide the gold layer into two separate regions, spaced 50 μm apart. A positive and negative lead was attached to each gold side and connected to the Harvard Apparatus BTX device.

Efficacy of the device was tested in the presence of yeast. The device was configured to deliver 20 pulses, with pulse lengths of 100 μs, 500V and 4 Hz. 1 ml of yeast, cultured to stationary phase in LB broth, was added to 10 μl of trypan blue, a dye that selectively colors dead or dying cells. The treated sample, as shown in FIG. 7E indicates dying cells 780 in the vicinity of the 50 μm spacing 790 between the gold electrodes.

### Example 4

A LVHEF device was constructed in a “comb” configuration using gold electrodes deposited on a glass substrate as shown in FIG. 7F. The distance between each gold electrode in the comb configuration was 16.3 μm. Conductive aluminum tape was attached to either side (+ and −). The conductive tape was attached to a function generator for power.

Efficacy of the device was tested in the presence of E.coli. The device was configured to deliver continuous pulses over 6 hours, with pulse lengths of 100 μs, 1.4V and 4 Hz. 2 ml of E. coli, cultured to stationary phase in LB broth, was added to 100 ml sterile water and divided into two 50 ml aliquots: one treated with the device and one untreated as a control. Each aliquot was subjected to a circulation via stirring rods and covered in parafilm to prevent outside contamination. The treated sample experienced a 0.06 log reduction in E. coli as compared to the untreated control. Experiments were repeated twice.

### Example 5

### Electrodes on Cardboard

Cardboard is a multilayer structure as shown in FIG. 8. The application of LVHEF electrodes may require a combination of electrical conductance parts and electrical insulation parts in such a way that the electric field is developed inside the fluid. The same concept of mutilayered structure as that described for the cardboard could be used with layers of plastic materials instead of paper.

Non Conductive Paper, Conductive Electrode and Non-Conductive Coating

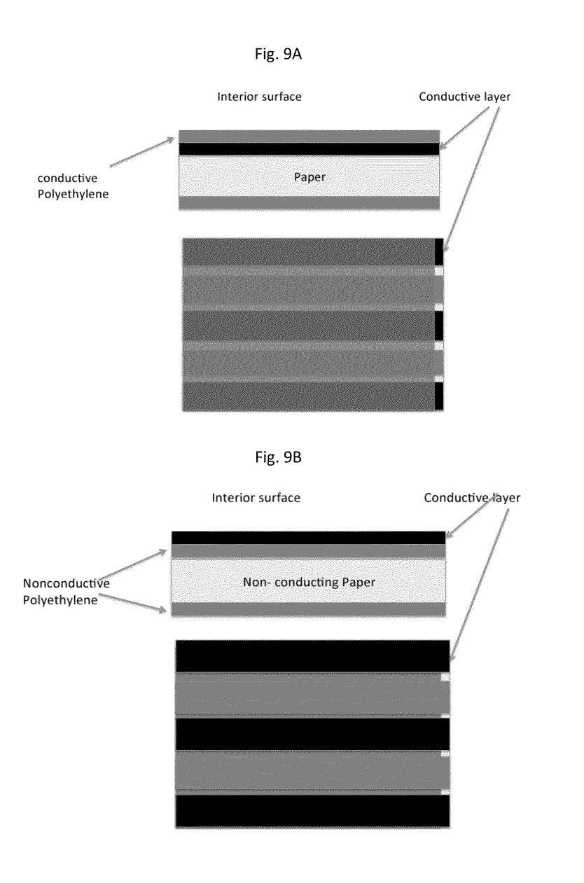

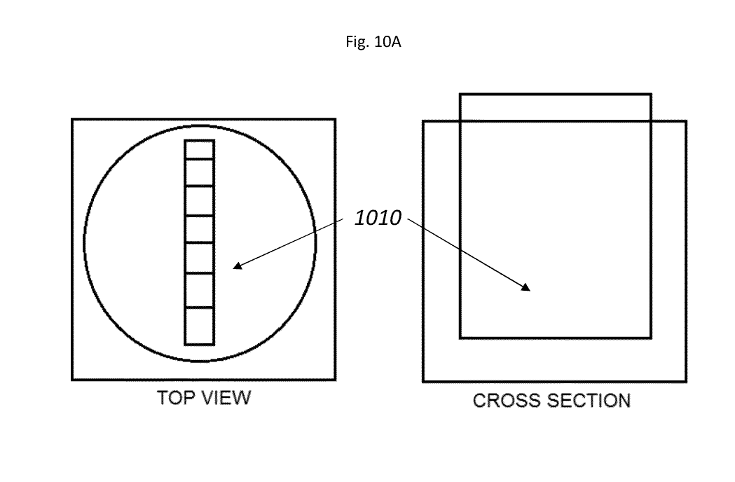

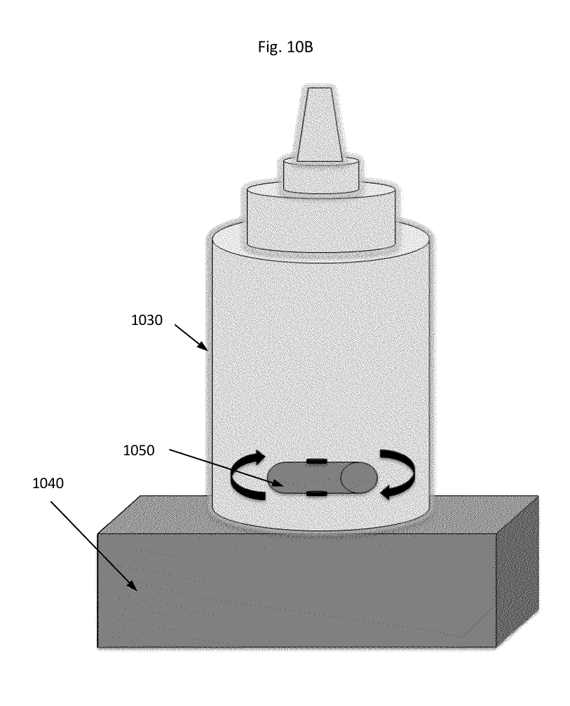

FIG. 9A illustrates a configuration of the walls of the container in which traditional paper is modifying by adding a layer of conductive material. The coating is non-conductive and the design is for an LVHEF configuration (but could be also for a single layer). This configuration requires an AC current.

The electric field in this circuit, in which a plastic coating is a dielectric with capacitance C1 and C2 on top of each electrode and the resistances are of the two electrodes R1 and R3 and the matter inside the container R2.

The mathematical analysis of the design is given below. This circuit may be described by Kirchhoff's law.

The complex impedance, Z, of the circuit is given by:

$$Z = R 1 + \frac{1}{j ω C 1} + R 2 + \frac{1}{jω C 2} + R 3$$

$$Where j = \sqrt{- 1} and ω = 2 π f$$

From the analysis of the circuit it is possible to find that when the voltage across the entire circuit is V, than the voltage across the matter in the container with resistance R2 is given by V_{R2}:

$${V}_{R 2} = \frac{V}{R 1 + \frac{1}{jω C 1} + R 2 + \frac{1}{jω C 2} + R 3} \cdot R 2$$

From equations of this type it is possible to calculate the design parameters. The potential required is proportional to R2, meaning that in the design, one would need a much lower potential. Because the coating can be made thin, the capacitance of the system is also low. This configuration requires AC currents. Therefore the system design can be such that the wavelength of the AC current can range from 10⁻⁹ to 10⁻³ sec. This LVHEF design, which requires low voltages, is accomplished even with a battery or a conventional function generator.

Non Conductive Paper, Conductive Electrode (EM) and Conductive Coating

In some variations, conductive polymer coating is used to coat electrodes. Primarily imbedding conductive particles such as metals, carbon nanotubes, or graphene in the polymer produces it. CoolPoly® E2 Thermally Conductive Liquid Crystalline Polymer (LCP) CoolPoly E series of thermally conductive plastics transfers heat, a characteristic previously unavailable in injection molding grade polymers. CoolPoly is lightweight, net shape moldable and allows design freedom in applications previously restricted to metals. The E series is electrically conductive and provides inherent EMI/RFI shielding characteristics. It should be preferable to choose a conductive polymer with the properties of the matter in the container.

Non Conductive Paper, Non-Conductive Coating and Conductive Electrode

As in FIG. 9B, the conductive element is in direct contact with the stored material. It is possible to obtain this design also by printing with conductive ink or using materials that do not contaminate the product such as gold or graphene. Graphene ink is used where suitable.

Conductive Paper, Non-Conductive Coating and Conductive Electrode

Paper can be also made conductive through various conductive additives to the pulp such as copper, graphene, carbon nanotubes. Adding carbon nanotubes or carbon nanofibers, commercially available paper is made highly conductive, with sheet resistances being reported as low as 1 ohm per square. Similar results are also obtained with graphene additives to paper (and other compounds in the board (coatings), such as those commercially available from XG Sciences, Inc.

Conductive and nonconductive paper, conductive and nonconductive coating and conductive electrodes of different kinds can be used in a large variety of configurations, of which those shown here are only illustrative examples. Further, in some aspects, a layer of plastic material may be used in place of, or in combination with, cardboard or paper products.

### Example 6

### LVHEF in Milk or Juice Containers

LVHEF electrodes are incorporated into a container for milk or juice. Electrodes are embedded in an alternating configuration and are powered by a small battery supply.

An order of magnitude estimate for the electric field strength A (V/cm) is evaluated from the equation below in which V (V) is the voltage between the electrodes and d (cm) is the distance between the electrodes.

A=V/d

For example, if an electric field of 10 kV/cm is required for killing cells with irreversible electroporation on the surface of the container with electrodes on the surface, then a gap of 0.5 micron would require a voltage of 0.5V between the electrodes. In contrast, to achieve an electric field of 10 kV/cm with an industrial scale distance between electrodes of 1 cm, a voltage of 10,000 V needs to be applied between the electrodes.

### Example 7

### Flow Mechanism with a Revolving Paddle

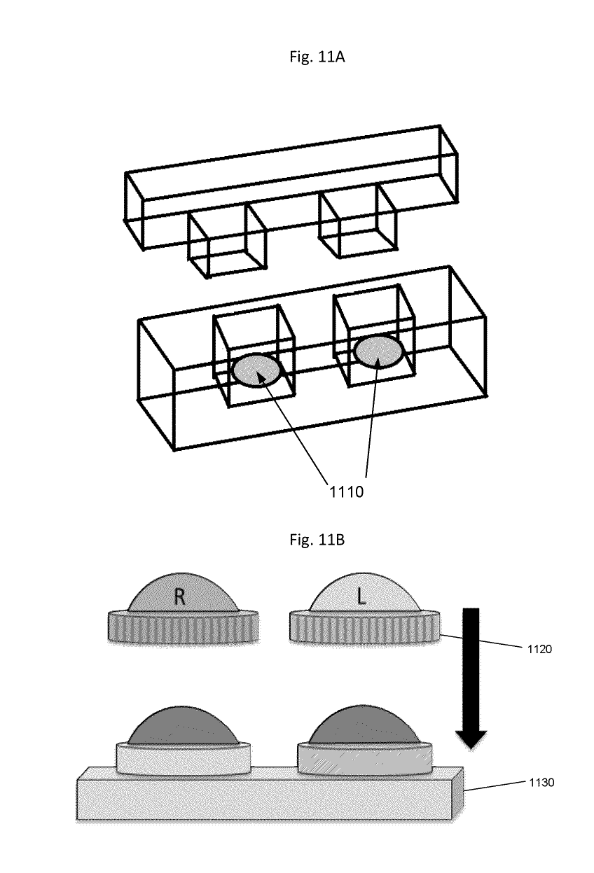

A container is designed with a revolving shaft attached to a propeller or paddle, on which LVHEF electrodes are installed. The shaft or paddle is configured with LVHEF electrodes on at least one surface, which is inserted in the material to be treated. The shaft is rotated around an axis such that the electrode surface rotates in the matter causing a flow within the container which induces mixing as well as bringing the matter to the surface with the electrodes. The surface with the electrodes rotating around the shaft can be configured with paper for single use or a solid plastic or metal for repeated uses. The power supply for the electrodes and for the rotation can be either manual, or from a battery or a wall plug and an electrical rotating motor. An example configuration is shown in FIG. 10A, with a rotating shaft and paddle 1010.

### Example 8

### Flow Mechanism with Stir Bar

A LVHEF insert is designed for storage of vaccine with a rotating stir bar containing electrically conductive gold LVHEF electrodes. The vaccine storage container is designed such that it could be used in conjunction with a small magnetic plate or induction plate. The stir bar is able to rotate upon an axis, powered by small batteries held in the stir bar itself, or by a magnetic plate. Rotation of the stir bar allows for flow in the container as shown in an example configuration in FIG. 10B, with a container 1030, a stir means 1050 and a magnetic plate 1040. Alternate configurations include eye drop solution containers and contact lens solution containers.

### Example 9

### LVHEF Contact Lens Container

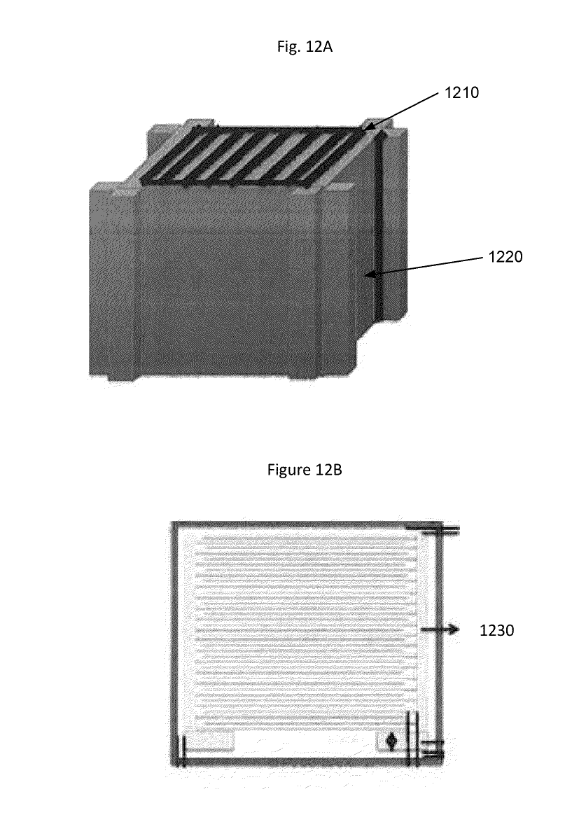

LVHEF electrodes are configured for a contact lens container as shown in FIG. 11A and FIG. 11B. Fine gold metal electrodes are configured in an alternating concentric arrangement around the walls and lids of each contact lens container space. Electrodes are embedded in the plastic and powered by a small 5V battery in the interior of the case. Electrodes are spaced 100 μm apart by polyethylene insulation and provide 100 μs pulses at 4 Hz. A small pump induces fluid flow into each container, circulating contact lens solutions around the electrodes. A configuration with lids 1120 and base 1130 is shown in FIG. 11B. The contact lens container also contains an LED which projects a timer for how long the solution has been exposed to electrode pulses and how many times each contact lens 1110 has been worn. LVHEF contact lens cases are able to reduce microorganism contamination in contact lens solution held within in the container for up to 2 days.

Another variation of the device provides for the contact lens case to be power by an induction unit, similar to a base found for an electric toothbrush. The contact lens case contains a rechargeable battery. When the case is placed on an induction unit, the base may be re-charged, allowing electrodes to deliver pulses for as long as there is charge in the battery.

Another variation of the device provides for the contact lens case to be powered only when the lids of the container are closed.

### Example 10

### LVHEF Insert for a Contact Lens Solution Bottle

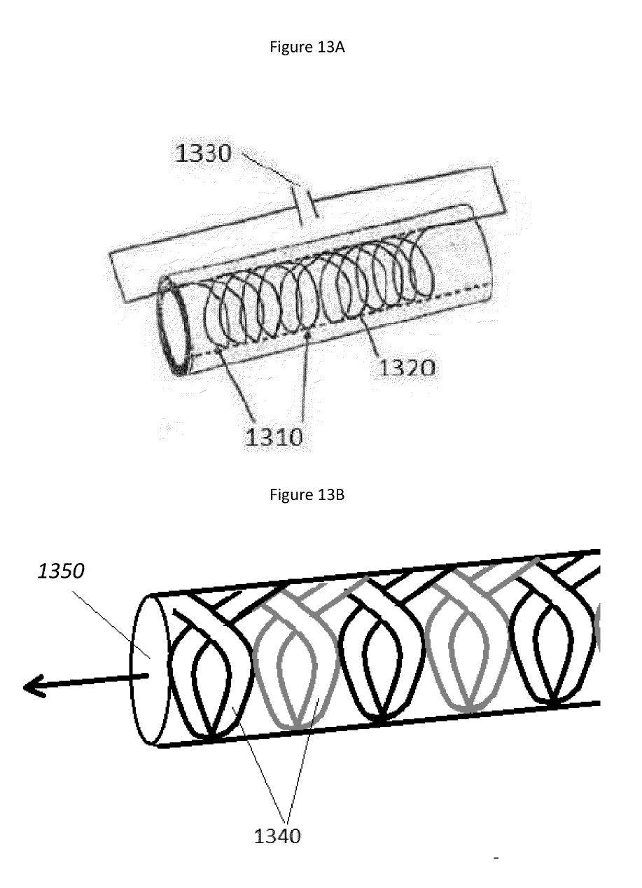

A LVHEF insert device is configured for sterilization of a container or bottle used for the storage of contact lens solution as shown in FIG. 12A. An insert 1220 is designed as a cube measuring about 7 mm×7 mm×7 mm and is attached to a base unit. The cube is configured such that surfaces of the cube are covered with fine gold metal electrodes 1210 and 1230 arranged in an inter-digitated pattern one or more sides of the cube as shown in FIG. 12B. Individual electrodes are spaced apart. The electrode covered portion of the insert is attached to a base unit containing a small 5V battery and a motor that drives the rotation of the cube. Rotation of the cube produces fluid flow in the container. The battery of the base unit also powers the electrodes to kill microorganism contamination in the storage bottle. The insert is deposited into bottles of contact lens solution and is able to reduce or prevent contamination of solution for up to 2 years. In various designs, the insert floats or is not otherwise attached to the container.

Another variation of the insert provides for the insert to be power by an induction unit, similar to a base found for an electric toothbrush. The insert contains a rechargeable battery. When the bottle containing the insert is placed on an induction unit, the battery inside the base may be re-charged, allowing electrodes on the insert to deliver pulses for as long as there is charge in the battery.

### Example 11

### LVHEF Electrodes for Embedded in a Beverage Distribution Pipe

LVHEF electrodes are configured in a serpentine arrangement on the inner surface of the walls of a pipe used in distribution and packaging of beverages. Titanium electrodes are configured in a serpentine arrangement on the inner walls of the pipe which has a diameter of at least 0.25 m. The electrodes are configured such that they are spaced about 200 μm apart and cover a substantial portion of the inner wall of the pipe. Electrodes are connected to an external power supply found outside of the pipe.

### Example 12

### Flexible LVHEF Electrodes for Insertion into a Tube

LVHEF electrodes 1310 are printed on a sheet of flexible plastic material 1320 in an inter-digitated manner. Fine gold electrodes spaced 500 nm apart are deposited on the pliable plastic sheet, as shown in FIG. 13A-B. The electrode containing sheet 1340 is then rolled and inserted into a dialysis tube 1350 and further attached to an external power source 1330. Electrodes produce LVHEF pulses which substantially reduce the threat of microbial contamination during fluid flow-through, such as during a dialysis procedure. In an alternate example, the electrodes and/or surface are non-flexible.

### Example 13

### LVHEF Electrodes on a Rolling Cylinder Extruder

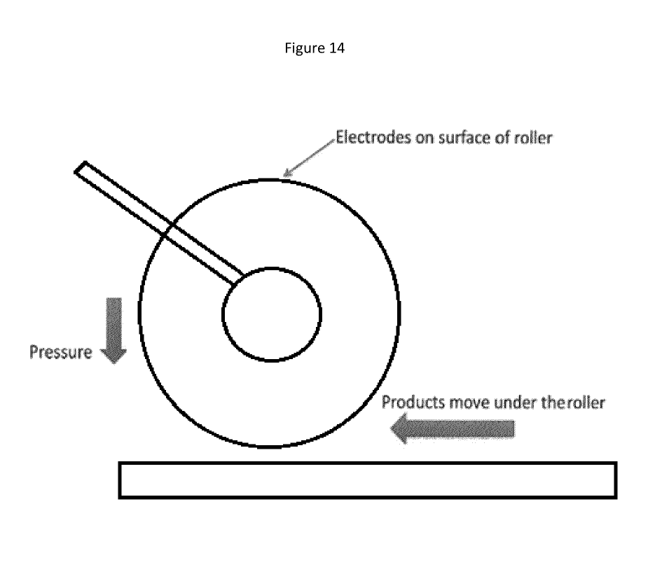

LVHEF electrodes are coated on a rolling cylinder extruder used in the formation and pressing of paper sheets used for packaging containers as shown in FIG. 14. Wood pulp and other base materials used for paper are processed by continuous pressing by large metal rolling cylinders. Cylinders produce pressure to flatten out thinner and thinner sheets of paper. Before and during this process, wood pulp and precursor materials may contain microbial contamination. Gold LVHEF electrodes are configured on a roller, such that paper pulp is contacted by electrodes during processing. LVHEF electrodes are coated on the surface of the roller and are connected to an external power source. LVHEF electrodes on rollers provide electric fields and pressure to reduce microbial contamination on the paper, while avoiding damage to the paper during the process.

### Example 14

### LVHEF Electrodes on a Conveyor Belt for Meat Patties

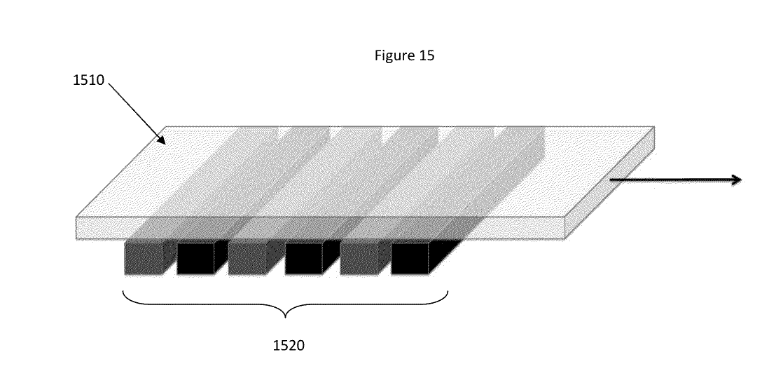

LVHEF electrodes 1520 are configured in an alternating co-planar or co-linear pattern underneath a thin moving conveyor belt used to transport products 1510 such as meat patties during processing and packaging. Before and after this process, meat and other patty materials may contain microbial contamination. Titanium LVHEF electrodes are configured such that meat patties on the conveyor belt are exposed to electric fields, provided by the LVHEF electrodes, as the patties move along the belt. LVHEF electrodes are connected to an external power source and provide electric fields with sufficient energy to kill a variety of microorganisms that may be found on the meat patties or on the conveyor belt. LVHEF electrodes are able to reduce microbial contamination on the meat, preventing contamination and spoilage, while avoiding cooking the meat. An example configuration of the conveyor belt is show in FIG. 15

While preferred embodiments of the present invention have been shown and described herein, such embodiments are provided by way of example only. Numerous variations, changes, and substitutions are envisioned without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for affecting cells in a volume of matter, said method comprising:
i) exposing a portion of the volume to one or more electric fields that are sufficient to kill at least a portion of exposed cells,
ii) mixing the exposed portion of the volume with an unexposed portion of the volume to form a mixed volume, and
iii) exposing a portion of the mixed volume to one or more electric fields that are sufficient to kill at least a portion of exposed cells;
wherein said volume of matter is bounded by a surface having one or more electrodes.

2. The method of claim 1, wherein said steps i)-iii) occur continuously or sequentially.

3. The method of claim 1, wherein said steps i) and iii) occur sequentially, and step ii) occurs continuously.

4. The method of claim 1, wherein said cells are microorganisms.

5. The method of claim 1, wherein said cells are selected from unicellular microorganisms, multicellular organisms, bacteria, parasites, fungi, protists, algae, larvae, nematodes, worms, and combinations thereof.

6. The method of claim 1, wherein said cells are bacteria.

7. The method of claim 1, wherein said matter is selected from a liquid, gas, fluid, colloid, gel, aerosol, foam, emulsion, suspension, heterogeneous solie-liquid composition, solution, and mixtures thereof.

8. The method of claim 1, wherein said matter is aqueous.

9. The method of claim 1, wherein said matter is selected from a pharmaceutical composition, a cosmetic composition, food composition, and a contact lens solution.

10. The method of claim 1, wherein said mixing occurs through movement.

11. The method of claim 10, wherein said movement is selected from the group consisting of:
convection, mechanical agitation, vibration, stirring, electrical field driven flows, electrophoresis, dielectrophoresis, osmotic flow, electro-osmosis, turbulent flow, laminar flow, natural convection, diffusion, and combinations thereof.

12. The method of claim 1, wherein said portion of the volume of matter of steps i) and iii) is each individually selected from the group consisting of less than 95%, 90%, 85%, 80%, 75%, 70%, 65%, 60%, 55%, 50%, 45%, 40%, 35%, 30%, 25%, 20%, 15%, 10%, 5%, and 1% of total volume of matter.

13. The method of claim 1, wherein steps i) and iii) each individually result in an amount of reduction of microorganisms selected from the group consisting of at least 95%, 90%, 85%, 80%, 75%, 70%, 65%, 60%, 55%, 50%, 45%, 40%, 35%, 30%, 25%, 20%, 15%, 10%, 5%, and 1% reduction of microorganisms in the exposed portion of the volume of matter.

14. The method of claim 1, wherein said method results in an amount of reduction of microorganisms in a total volume of matter selected from the group consisting of at least 95%, 90%, 85%, 80%, 75%, 70%, 65%, 60%, 55%, 50%, 45%, 40%, 35%, 30%, 25%, 20%, 15%, 10%, 5%, and 1% reduction of microorganisms.

15. The method of claim 1, wherein said method is repeated over a period of time.

16. The method of claim 15, wherein said period of time is selected from up to 1 hour, 4 hours, 8 hours, 12 hours, 24 hours, 2 days, 3 days, 4 days, 5 days, 6 days, 7 days, 2 weeks, 3 weeks, 4 weeks, 2 months, 3 months, 4 months, 5 months, 6 months, a year, and two years.

17. The method of claim 15, wherein said method is repeated sequentially for a number of cycles selected from the group consisting of at least 1, 2, 3, 4, 5, 10, 20, 50, 100, 500, 1000, 10,000, 100,000, and 250,000 cycles.

18. The method of claim 1, wherein said surface having one or more electrodes forms part or all of a container encompassing said volume of matter.

19. The method of claim 1, wherein said surface having one or more electrodes forms part or all of a conduit for transporting said volume of matter.

20. The method of claim 19, wherein said conduit is a pipe.

21. The method of claim 1, wherein said surface having one or more electrodes forms a surface immersed in said volume of matter.

22. The method of claim 21, wherein said surface immersed in said volume of matter is in the form of an insert for a packaged material.

23. The method of claim 1, wherein said surface having one or more electrodes is in direct contact with the volume of matter.

24. The method of claim 1, wherein said a surface having one or more electrodes is in indirect contact with the volume of matter.

25. The method of claim 1, wherein said surface having one or more electrodes is contained in a medical device.

26. The method of claim 1, wherein said one or more electric fields range from 50 V/cm to 100 kV/cm.

27. The method of claim 1, wherein said electric fields are generated with low voltage.

28. The method of claim 1, wherein said electric fields are generated by a voltage selected from less than about 10,000V, 1000V, 100V, 10V, 1V, and 0.5V.

29. The method of claim 28, wherein said voltage is less than about 10V.

30. The method of claim 1, wherein said electric fields are generated with direct current (DC), alternating current (AC), or pulsed current or electromagnetic induction.

31. The method of claim 30, wherein said AC current has a wavelength selected from less than 10⁻² s, 10⁻³ s 10⁻⁴s, 10⁻⁵ s, 10⁻⁶ s, 10⁻⁷ s, 10⁻⁸ s, and 10⁻⁹ s.

32. The method of claim 30, wherein said pulsed electric fields are pulsed with a pulse length selected from a time less than 1 s, 10⁻¹ s, 10⁻² s, 10⁻³ s 10⁻⁴s, 10⁻⁵ s, 10⁻⁶ s, 10⁻⁷ s, 10⁻⁸ s, and 10⁻⁹ s.

33. The method of claim 30, wherein interval between said pulsed electric fields are selected from a time less than 100 s, 60s, 1 s, 10⁻¹ s, 10⁻² s, 10⁻³ s 10⁻⁴s, 10⁻⁵ s, 10⁻⁶ s, 10⁻⁷ s, 10⁻⁸ s, and 10⁻⁹ s.

34. The method of claim 30, wherein pulsed electric fields are a single polarity.

35. The method of claim 30, wherein pulsed electric fields are alternating polarity.

36. The method of claim 1, wherein said electric fields are substantially non-uniform throughout said volume of matter.

37. The method of claim 1, wherein at least a portion of exposed cells undergo electroporation.

38. The method of claim 37, wherein said electroporation is selected from the group consisting of reversible electroporation, irreversible electroporation, nanosecond pulses and combinations thereof.

39. The method of claim 1, wherein said surface has one electrode.

40. The method of claim 1, wherein said surface has at least 2 electrodes.

41. The method of claim 40, wherein said electrodes are separated by a gap.

42. The method of claim 41, wherein said gap is between 10 nanometers and 500 microns.

43. The method of claim 41, wherein said gap is between 10 nanometers and 50 microns.

44. The method of claim 41, wherein said gap is between 10 nanometers and 10 microns.

45. The method of claim 41, wherein said gap is between 10 nanometers and 100 nanometers.

46. The method of claim 41, wherein said gap comprises an insulator.

47. The method of claim 1, wherein one or more electrodes include a metal selected from the group consisting of Au, Pt, Cr, Cu, Al and Ti.

48. The method of claim 1, wherein one or more electrodes comprise a semi-conducting material.

49. The method of claim 1, wherein one or more electrodes are arranged in a coplanar configuration.

50. The method of claim 1, wherein at least one electrode comprises a layer of conductive material contacted by a layer of insulating material containing a plurality of gaps.

51. The method of claim 50, wherein said gaps have a dimension of less than about 500 microns.

52. The method of claim 50, wherein the gaps are spaced at about 100 microns apart.

53. The method of claim 1, wherein said surface and/or said electrodes are flexible.

54. The method of claim 1, wherein said surface further comprises charged particles or a charged polymer.

55. The method of claim 1, wherein one or more physical or chemical properties of the matter are not detectably altered by the method.

56. The method of claim 55, wherein said matter has a taste which is not detectably altered by the method when subjected to a human taste test.

57. The method of claim 55, wherein the matter has a medical function that is not detectible altered by the method when used for medical applications.

58. The method of claim 1, wherein the method does not raise a temperature for the volume of matter by more than 20° C.

59. The method of claim 1, wherein microorganisms in said exposed portion of the volume have a reduced ability to reproduce.

60. A method for increasing the shelf-life of a perishable composition comprising the step of preparing a perishable composition and performing the method of claim 1 on said perishable composition.

61. A method for treating contact lens solution comprising performing the method of claim 1, wherein said matter is a solution for storage of contact lenses.

62. A container, apparatus, pipe or device configured to perform the method of claim 1.

63. A container according to claim 62, wherein said container is further configured to enclose a food or beverage.

64. A container according to claim 62, wherein said container is configured to enclose a solution or object selected from the group consisting of a pharmaceutical agent, a medical agent, a medical device, a cleaning solution, a food, and a beverage.

65. A device according to claim 62, wherein said device is configured as an insert in a solution or object selected from the group consisting of a pharmaceutical agent, a medical agent, a medical device, and a cleaning solution.

66. A device according to claim 62, wherein said device is configured as an insert in a food or beverage container.

67. A composition of matter, wherein said composition is treated according to the method of claim 1.

68. The composition according to claim 67, wherein said composition is selected from the group consisting of food, beverage, cosmetic, and pharmaceutical.

69. A method for affecting cells in a macroscopic volume of matter, said method comprising:
i) contacting said volume of matter with a surface having one or more electrodes and exposing a portion of the volume to one or more electric fields that are sufficient to kill at least a portion of exposed cells,
ii) moving the exposed portion of the volume with respect to said surface having one or more electrodes, or moving said surface having one or more electrodes with respect to the exposed portion of the volume, and
iii) contacting a previously unexposed portion of said volume of matter with a surface having one or more electrodes and exposing said previously unexposed portion of the volume to one or more electric fields that are sufficient to kill at least a portion of exposed cells.

70. The method of claim 69, wherein said electric fields are substantially non-uniform throughout said volume of matter.

71. The method of claim 69, further comprising application of pressure to the volume of matter during one or more steps of i-iii.

72. The method of claim 69, wherein the volume of matter is selected from solids and glass.
